(19) United States
(12) Patent Application Publication
(10) Pub. No.: US 2023/0086008 A1
(43) Pub. Date: Mar. 23, 2023

(54) ENGINEERED MICROBIAL POPULATION DYNAMICS FOR BIOSENSING AND INFORMATION PROCESSING

(71) Applicant: Inscripta, Inc., Boulder, CO (US)

(72) Inventor: Tyson Shepherd, Boulder, CO (US)

(21) Appl. No.: 17/683,692

(22) Filed: Mar. 1, 2022

Related U.S. Application Data
(60) Provisional application No. 63/156,712, filed on Mar. 4, 2021.

(51) Int. Cl. C12Q 1/689; C12N 15/10; G16B 20/20

(52) U.S. Cl. CPC C12Q 1/689; C12N 15/1065; G16B 20/20; C12N 2310/20

(57) ABSTRACT

The present disclosure relates to cell populations and systems for detection of compounds in an environment. Specifically the disclosure relates to methods for generating reproducible genome-wide edited populations of microbes that display novel, defined, and reproducible phenotypes when exposed to one or more chemicals. In some applications, such phenotypes are read out by barcode amplicon and compared against population fingerprints. In other applications digital information is stored in such populations of microorganisms. The digital information can be retrieved from the microorganisms with Boolean logic.

### CROSS-REFERENCE

The present application claims priority to U.S. Provisional Application Ser. No. 63/156,712, filed Mar. 4, 2021, the contents of which are hereby incorporated by reference in their entirety.

### SEQUENCE LISTING

The application contains a Sequence Listing, which has been filed electronically in ASCII format and is hereby incorporated by reference in its entirety. Said ASCII copy, created on Sep. 22, 2022, is named INSC082US_ST25.txt and is 12,288 bytes in size as measured in Microsoft Windows®.

### FIELD OF THE INVENTION

The present disclosure relates to automated multi-module instruments, compositions and methods for performing nucleic acid-guided nuclease editing; specifically, the present disclosure relates to methods for generating engineered microbial population dynamics for biosensing and digital information processing.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

DNA is a polymer used for storage and transmission of genetic information in biology. In principle, DNA can also be used as a medium for the storage of arbitrary digital information at densities exceeding existing capabilities for commercial data storage and at scales well beyond the capacity of current data centers. Nonetheless, the requirements for nucleic acid synthesis (e.g., writing of the information) and the limitations of the technologies available for sequencing the DNA (e.g., reading of the information) remain significant challenges in rendering DNA-based digital information storage potentially viable economically.

The ability to make precise, targeted changes to the genome of living cells has been a long-standing goal in biomedical research and development. Recently various nucleases have been identified that allow manipulation of gene sequence, and hence gene function. The nucleases include nucleic acid-guided nucleases, which enable researchers to generate permanent edits in live cells, as well as to introduce exogenous information more efficiently into cells.

There is thus a need in the art of nucleic acid-guided nuclease gene editing for improved methods, compositions, modules and instruments for introducing, storing, and retrieving high density information into cells. The present invention satisfies this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In some aspects, the present disclosure provides compositions, automated methods and multi-module automated instrumentation for high throughput screening and selection of microbes that are either resistant or tolerant to a wide variety of small molecule compounds.

The present disclosure thus provides, in selected embodiments, modules, instruments, and systems for automated multi-module cell editing for generating cell populations having properties desirable for positive and negative selection screens. Other specific embodiments of the automated multi-module cell editing instruments of the disclosure are designed for recursive genome editing, e.g., sequentially introducing multiple edits into genomes inside one or more cells of a cell population through two or more editing operations within the instruments. The sequential editing can provide for unique cell populations with defined phenotypes for use in positive and negative selective screens.

In specific cases, the disclosure describes useful applications of a population of cells having engineered phenotypes that enable selection in a positive selection screen (e.g. a tolerance to a compound), a negative selection screen (e.g. a resistance to a compound), or a combination of both.

Such cell populations can be engineered to store terabytes of exogenous digital information encoded as nucleic acids. The digital information can be retrieved with AND/OR gate logic based on the presence or absence of a small molecule compound or small molecule compounds of interest by parallel or serial passage of the cell populations in the compounds. Thus Boolean logic could be generated by selections on the pools of microbial cells through selection of engineered cell populations in compounds of choice.

In other cases, such cell populations can be engineered to identify an environmental pollutant, such as, for instance a presence of lead or another heavy metal in a water sample.

The disclosure includes methods of using nucleic-acid guided editing in cell populations, e.g., bacterial and fungal cells, for creation of cell populations that can be selected for with a compound of choice. In some aspects, the disclosure comprises methods to identify cells edited with a library of vectors that have unique multiplex sets of mutations—conferring microbial resistance or microbial sensitivity to a compound. In particular, the disclosure provides methods for identifying edited cells that have increased tolerance/sensitivity to non-antibiotic compounds, an effective alternative to antibiotic selection systems. In addition, the disclosure provides methods for generating engineered cells for storage of large amounts of digitally encoded information encoded in vectors transformed into these cells.

In some aspects, the cells generated using the methods of the disclosure have a series of multiplex genetic variations that effectively support a use in positive and/or negative selection systems.

In some aspects the disclosure provides a cell population genetically engineered to have a tolerance to a first compound and a sensitivity to a second compound by genome wide editing with a library of CRISPR editing cassettes. The library of gene editing cassettes can be a library of randomly designed CRISPR editing cassettes or a library of rationally designed CRISPR editing cassettes. The CRISPR editing cassettes can be, e.g., CREATE gene editing cassettes comprising: a nucleic acid encoding a guide RNA (gRNA) comprising a region complementary to a target region of a nucleic acid in one or more cells of the cell population; a region homologous to the target region in the one or more cells of the cell population having a change in sequence of at least one nucleotide relative to a wild type reference genome of the cell; and a region comprising a site conferring immunity to nuclease-mediated editing.

In some aspects, the present disclosure provides a method for Boolean storage of information in a cell population comprising: transforming a cell population genetically engineered to have a tolerance to a first compound and a sensitivity to a second compound with a plurality of nucleic acids encoding at least two digital information files, thereby storing the at least two digital information files in a transformed cell population having Boolean logic for information retrieval. In some instances, the method further comprises growing the cell population having the at least two digital information files in the first compound and in the second compound in parallel (OR) or in series (AND) for selecting cell populations having Boolean selections of one or more cells encoding the at least two digital information files. In preferred cases, the cell population is engineered to have a tolerance to at least 2 or more compounds, such as a tolerance to coumaric acid, isobutryic acid, furfural, vanillin, resveratrol, 5-hydroxymethyl-furfural, NaCl, or syringic acid. Alternatively, the cell population can be engineered to have a sensitivity to at least 2 or more compounds, such as a sensitivity to coumaric acid, isobutryic acid, furfural, vanillin, resveratrol, 5-hydroxymethyl-furfural, NaCl, or syringic acid. In preferred cases, the method offers an alternative to traditional antibiotic methods for cell selection, and neither the first compound nor the second compound are antibiotics.

Further, the method provides for digital information storage of any content that can be written into a digital language. In preferred instances at least two digital information files do not encode a component of a biological pathway, in other words, the content written into the cell does not encode biological information. In some instances, the method further allows for the retrieving of the at least two digital information files from the cell populations having Boolean logic for information retrieval by sequencing, such as whole genome sequencing or the sequencing of a plasmid. In preferred cases, the cell population comprises a barcode for sequencing at least one digital information file from the at least two digital information files to facilitate retrieval of the information. The barcode can be, e.g., on a plasmid vector that is incorporated into the cell population. In such instances, the cell population itself has been genetically modified/selected to have a tolerance or a sensitivity to a compound—as opposed to a plasmid vector conferring the tolerance or the sensitivity. In some instances, the plurality of nucleic acids encoding the exogenous digital information file can encode up to 50 million gigabytes, up to 100 million gigabytes, up to 150 million gigabytes, or up to 200 million gigabytes of the digital information encoding a plurality of exogenous digital information files, e.g., files encoding redundant information or files encoding distinct information. The exogenous digital information file may be first written as a sequence of 0s and 1s, and these 0s and 1s can be written into the plurality of nucleic acids with either the nucleotide bases, A, C, G, or T encoding for either 0 or 1. In some aspects the disclosure provides a population of cells having Boolean logic for information retrieval as described above.

In other aspects, the disclosure provides a method of environmental biosensing comprising: contacting a sample from an environment to a cell population genetically engineered to have a tolerance to a plurality of compounds, wherein one or more cells in the cell population are associated with at least one barcode, whereby each barcode is a unique identifier for a compound in the plurality of compounds; enriching for one or more cells in the cell population by growing the one or more cells in the presence of the sample from the environment, thereby generating a sample tolerant population; and sequencing the at least one barcode from one or more cells in the sample tolerant population thereby generating a sequencing file that identifies a cell having a tolerance to the sample, whereby the identification of the tolerant cell provides for environmental biosensing.

The cell populations engineered with the methods of the disclosure can be engineered to have a tolerance to at least 5 compounds, at least 100 compounds, or at least 500 compounds. In some cases, the cell population is genetically engineered to have a tolerance to a first pre-determined compound, such as coumaric acid, isobutryic acid, furfural, vanillin, resveratrol, 5-hydroxymethyl-furfural, NaCl, or syringic acid. The first pre-determined compound can be an environmental pollutant and in preferred cases the cell population is not engineered to have a tolerance to an antibiotic. The cell population may be further engineered to have a sensitivity to a plurality of compounds, e.g., engineered to have a sensitivity to at least 5 compounds, at least 100 compounds, or at least 500 compounds. In some cases, the cell population is genetically engineered to have a sensitivity to a second pre-determined compound, such as coumaric acid, isobutryic acid, furfural, vanillin, resveratrol, 5-hydroxymethyl-furfural, NaCl, or syringic acid. The second pre-determined compound can be an environmental pollutant and in preferred cases it Is not an antibiotic.

In additional aspects, the disclosure provides methods for comparing the growth of the sample tolerant/sensitive population against a population fingerprint, wherein the population fingerprint is a reference population growth behavior in the presence of a known compound.

In specific aspects, the disclosure provides cells produced by the disclosed methods, where the cells are genetically engineered to have a sensitivity or a tolerance to one or more compounds. In other aspects, the disclosure provides cells produced by the disclosed methods, where the cells are designed to efficiently store large amounts of digital information.

The cells that can be edited using the methods of the disclosure include any prokaryotic, archaeal or eukaryotic cell. For example, prokaryotic cells for use with the present illustrative embodiments can be gram-positive bacterial cells or gram-negative bacterial cells, e.g., E. coli cells. Eukaryotic cells for use with the automated multi-module cell editing instruments of the illustrative embodiments include any plant cells and any animal cells, e.g. fungal cells (including yeast), insect cells, amphibian cells, and the like.

In some aspects, the microbes edited using the methods of the disclosure include bacterial cells. In some aspects, the microbes edited using the methods of the disclosure include fungal cells.

In preferred aspects, the digital information is introduced as heterologous sequences into microbial systems. The genes could either be integrated into the new host genome or carried on a plasmid.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A is an illustrative schematic for generating a population of microbes that carry information segmented across populations that have been raised to have tolerance to a set of exemplary compounds. FIG. 1B illustrates a fingerprint molecular population behavior of E. coli populations raised to be tolerant/sensitive to coumaric acid and isobutyric acid.

FIGS. 2A-2C depict three different views of an exemplary automated multi-module cell processing instrument for performing nucleic acid-guided nuclease editing.

FIG. 3A depicts one embodiment of a rotating growth vial for use with the cell growth module described herein and in relation to FIGS. 3B-3D. FIG. 3B illustrates a perspective view of one embodiment of a rotating growth vial in a cell growth module housing. FIG. 3C depicts a cut-away view of the cell growth module from FIG. 3B. FIG. 3D illustrates the cell growth module of FIG. 3B coupled to LED, detector, and temperature regulating components.

FIG. 4A depicts retentate (top) and permeate (middle) members for use in a tangential flow filtration module (e.g., cell growth and/or concentration module), as well as the retentate and permeate members assembled into a tangential flow assembly (bottom). FIG. 4B depicts two side perspective views of a reservoir assembly of a tangential flow filtration module. FIGS. 4C-4E depict an exemplary top, with fluidic and pneumatic ports and gasket suitable for the reservoir assemblies shown in FIG. 4B.

FIGS. 5A and 5B depict the structure and components of an embodiment of a reagent cartridge. FIG. 5C is a top perspective view of one embodiment of an exemplary flow-through electroporation device that may be part of a reagent cartridge. FIG. 5D depicts a bottom perspective view of one embodiment of an exemplary flow-through electroporation device that may be part of a reagent cartridge. FIGS. 5E-5G depict a top perspective view, a top view of a cross section, and a side perspective view of a cross section of an FTEP device useful in a multi-module automated cell processing instrument such as that shown in FIGS. 2A-2C.

FIG. 6A depicts a simplified graphic of a workflow for singulating, editing and normalizing cells in a solid wall device. FIGS. 6B-6D depict an embodiment of a solid wall isolation incubation and normalization (SWIIN) module. FIG. 6E depicts the embodiment of the SWIIN module in FIGS. 6B-6D further comprising a heater and a heated cover.

FIG. 7 is a simplified block diagram of an embodiment of an exemplary automated multi-module cell processing instrument comprising a solid wall singulation/growth/editing/normalization module for recursive cell editing.

FIG. 8A depicts plots showing the average of replicate log10 design representation (1DRs) of the growth inhibitor of each design on the y axis and average of replicate 1DRs of the control media of each design on the x axis, showing a distribution where each data point represents a single design and those data points that deviate significantly away from the best fit line are representative of the enrichers and depletors, being associated with some promoter-spacer-RBS insert or gene knock out.

FIG. 8B depicts a principal component analysis of the bulk response in the presence of different growth inhibitors. Placement of the library response in the decomposition indicates a response identifying that particular compound.

FIG. 8C depicts plots showing a Log10 Design Representation (1DR) of M9 barcode sequence counts on the x-axis and the titled inhibitor or media on the y-axis. Each point on the scatter plot is the average 1DR of triplicate measures in the media and is representative of a single design.

FIG. 8D illustrates the principal component analysis (PCA) of the bulk population responses to the presence of the inhibitor compounds.

It should be understood that the drawings are not necessarily to scale, and that like reference numbers refer to like features.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

Detection of compounds in the environment can be a time consuming and laborious task that is prone to error and it is often idiosyncratic to the targeted compound. The state-of-the-art techniques for detection of environmental compounds typically rely on mass spectrometry, chromatography, or a fluorescent or chromogenic marker. Biosensing provides a shift towards integrating detection of compounds into the environment to allow for detection of biological or chemical issues. Typically, these efforts have been focused on biochemical responses including RNA switch responses and genetic circuits that allow for signaling between the molecule and a response in an individual microbe in a population containing a similar genetic makeup. However, some compounds can have a significant lag time in developing a switch response to a particular chemical. Further, this strategy is idiosyncratic to each chemical, and is not scalable. Moreover, the readouts of these assays are often through fluorescent proteins that cannot give a readout on diverse chemical compositions.

In some aspects, provided herein are systems and methods for accessing information stored in genetic material from a pool of cells, such as information carried by select populations of microbes. In some cases, a population of microbes that have been engineered to be tolerant to a particular chemical. Subsequently, the information stored in the genetic material of the tolerant population is extracted, and in preferred cases, separated from other microbes in the pool. For example, a genetic population can be raised to have tolerance to, for example, vanillin and/or resveratrol and a second genetic population can be raised to have tolerance to, for example, furfural. For example, a genetic population can be raised to have tolerance to, for example, resveratrol and a second genetic population can be raised to have a sensitivity to, for example, resveratrol. A genetically diverse population can be raised in additional biomass inhibitor compounds with genetic tolerance signatures being determined by the population behavior in each compound using the CREATE technology or another suitable genetic engineering approach. For “CREATE” technology, see U.S. Pat. Nos. 9,982,278; 10,266,849; 10,240,167; 10,351,877; 10,364,442; 10,435,715; 10,465,207; 10,669,559; 10,711,284; and U.S. Ser. No. 16/550,092; and 16/773,712, all of which are incorporated by reference herein.

Strains identified as having tolerance to a compound can become addressable in nature through selection by transforming the resistant colony with a plasmid or set of plasmids encoding a region of DNA of interest that may include a gene sequence, or a DNA sequence that encodes digital information. Strains determined to have tolerance to a different compound can be separately addressable for selections and can be transformed with DNA of interest that includes separate gene sequences or separate DNA sequences that encode different digital information.

The present invention addresses these needs by leveraging a high throughput method of raising microbes to have a tolerance to a wide variety of small molecule compounds. The disclosure demonstrates that sets of populations of E. coli can be raised to be tolerant and sensitive to exemplary compounds, including furfural, vanillin, resveratrol, 5-methyl-hydroxyfurfural, syringic acid, coumaric acid, and isobutyric acid, among others.

A microbial strain that is sensitive to exemplary compounds described herein, e.g., vanillin, resveratrol, 5-methyl-hydroxyfurfural, syringic acid, coumaric acid, and isobutyric acid, is an alternative to a strain that is engineered to have an antibiotic sensitivity.

In some aspects, the disclosure provides automated multi-module instruments, compositions and methods for performing nucleic acid-guided nuclease editing in a population of microbial cells for generating a wide variety of selective growth inhibited populations. As described herein, such microbial cell populations can be enriched from a population out of a set of other populations containing other sets of information. Further, combining resistance genotypes together further improves both selection capacity, as well as create a method for AND/OR gate logic based on the presence or absence of the compound or compounds of interest by parallel or serial passage in the compounds. Thus Boolean logic could be generated by selections on the pools through subjecting them to compounds in order to query data from the pool by selective growth.

Describe herein is a genome-wide editing approach for promoter insertions, knockouts, and protein coding region modifications that can generate microbes tolerant to small molecule growth inhibitors that in wild-type cells would cause a slow-growth, deadly, or otherwise undesirable phenotype. These edits can have different phenotypic responses based on the small molecule that the microbe is grown in. For example, wild-type E. coli growth is inhibited in the presence of vanillin concentrations in rich media (e.g. 4 mg/ml) or, separately, in certain concentrations of furfural in rich media (e.g. 4 mg/ml). Bacterial strains can also be engineered to have different tolerances or sensitivities to resveratrol, see, e.g., Examples. For instance, some promoter insertions or coding sequence knockouts have shown that E. coli can be raised with mutations that provide tolerance or sensitivity to these compounds, such that some will grow faster in vanillin than wild type, some will grow slower in vanillin than wild type, whereas some will grow faster in furfural and some will grow slower in furfural compared to wild type. Some strains will have tolerance to both vanillin and furfural, whereas other strains may have tolerance to one and sensitivity to the other, or vice versa, and some strains will have sensitivity to both, but may have tolerance to a 3rd chemical. The methods of the disclosure allow for the generation and selection of populations that have similar phenotypic responses to a variety of other compounds.

In some respects, provided herein is: (1) A population of tolerant and sensitive engineered microbial variants that has acquired identifiable phenotypes when the microbe is grown in the presence of a small molecule. Thus, the engineered population can identify a compound based on its phenotypic response. Furthermore, the microbial variants can be engineered to also have a molecular barcode, and thus the barcoded population trends can identify a compound in a solution through next generation sequencing, as a biosensor readout on the presence or absence of a compound. In other respects, provided herein is a (2) A population of tolerant strains to 1, 2, 3, or more than 3 compounds can be made to provide a digital (on/off) selectable and combinatorial marker for retrieval of stored genetic or digital information.

The practice of the techniques described herein may employ, unless otherwise indicated, conventional techniques and descriptions of molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th, ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, New York, 1989; and Chang, et al., Guide to Electroporation and Electrofusion, Academic Press, California (1992), all of which are herein incorporated in their entirety by reference for all purposes. Nucleic acid-guided nuclease techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

The term “biosensing” as used herein refers to the detection of biomolecules using the engineered cell populations of the disclosure (i.e., biosensor) that combines a biological component with a “read-out” mechanism (i.e., sequencing of the cell population—targeted sequencing, barcoded sequencing, whole genome sequencing or any other suitable method). The read-out mechanism may also be a colorimetric change in the cell, i.e., a change in yeast color from white to yellow, red, or blue in the presence of a target compound.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

As used herein the term “donor DNA” or “donor nucleic acid” refers to nucleic acid that is designed to introduce a DNA sequence modification (insertion, deletion, substitution) into a locus (e.g., a target genomic DNA sequence or cellular target sequence) by homologous recombination using nucleic acid-guided nucleases. For homology-directed repair, the donor DNA must have sufficient homology to the regions flanking the “cut site” or site to be edited in the genomic target sequence. The length of the homology arm(s) will depend on, e.g., the type and size of the modification being made. The donor DNA will have two regions of sequence homology (e.g., two homology arms) to the genomic target locus. Preferably, an “insert” region or “DNA sequence modification” region—the nucleic acid modification that one desires to be introduced into a genome target locus in a cell—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the genomic target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the genomic target sequence.

As used herein, “enrichment” refers to enriching for edited cells by singulation, inducing editing, and growth of singulated cells into terminal-sized colonies (e.g., saturation or normalization of colony growth).

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease. The term “editing gRNA” refers to the gRNA used to edit a target sequence in a cell, typically a sequence endogenous to the cell. The term “curing gRNA” refers to the gRNA used to target the curing target sequence on the editing vector.

“Homology” or “identity” or “similarity” refers to sequence similarity between two peptides or, more often in the context of the present disclosure, between two nucleic acid molecules. The term “homologous region” or “homology arm” refers to a region on the donor DNA with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

As used herein, the terms “protein” and “polypeptide” are used interchangeably. Proteins may or may not be made up entirely of amino acids.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible, and in some embodiments particularly many embodiments such as those described herein—the transcription of at least one component of the nucleic acid-guided nuclease editing system—and typically at least three components of the nucleic acid-guided nuclease editing system—is under the control of an inducible promoter.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, nourseothricin N-acetyl transferase, chloramphenicol, erythromycin, tetracycline, gentamicin, bleomycin, streptomycin, rifampicin, puromycin, hygromycin, blasticidin, and G418 may be employed. In other embodiments, selectable markers include, but are not limited to sugars such as rhamnose, human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+ cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2a; detectable by Mab-FITC); Methylguanine-DNA methyltransferase (MGMT; selectable by carmustine); and Cytidine deaminase (CD; selectable by Ara-C). “Selective medium” as used herein refers to cell growth medium to which has been added a chemical compound or biological moiety that selects for or against selectable markers.

The term “specifically binds” as used herein includes an interaction between two molecules, e.g., an engineered peptide antigen and a binding target, with a binding affinity represented by a dissociation constant of about 10′ M, about 10⁻⁸M, about 10⁻⁹ M, M, about 10⁻¹⁰ In about 10⁻¹¹M, about 10⁻¹² M about 10⁻¹³M, about 10⁻¹⁴M or about 10⁻¹⁵M.

The terms “target genomic DNA sequence”, “cellular target sequence”, or “genomic target locus” refer to any locus in vitro or in vivo, or in a nucleic acid (e.g., genome) of a cell or population of cells, in which a change of at least one nucleotide is desired using a nucleic acid-guided nuclease editing system. The cellular target sequence can be a genomic locus or extrachromosomal locus. The term “curing target sequence” refers to a sequence in the editing vector that is cleaved or cut to cure or clear the editing vector. The term “target sequence” refers to either or both of a cellular target sequence and a curing target sequence.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like. As used herein, the phrase “engine vector” comprises a coding sequence for a nuclease to be used in the nucleic acid-guided nuclease systems and methods of the present disclosure. The engine vector may also comprise, in a bacterial system, the λ Red recombineering system or an equivalent thereof. Engine vectors also typically comprise a selectable marker. As used herein the phrase “editing vector” comprises a donor nucleic acid, including an alteration to the cellular target sequence that prevents nuclease binding at a PAM or spacer in the cellular target sequence after editing has taken place, and a coding sequence for a gRNA. The editing vector may also and preferably does comprise a selectable marker and/or a barcode. In some embodiments, the engine vector and editing vector may be combined; that is, all editing and selection components may be found on a single vector. Further, the engine and editing vectors comprise control sequences operably linked to, e.g., the nuclease coding sequence, recombineering system coding sequences (if present), donor nucleic acid, guide nucleic acid(s), and selectable marker(s).

### Nuclease-Directed Genome Editing Generally

In preferred embodiments, the automated instrument described herein performs recursive nuclease-directed genome editing methods for introducing edits to a population of cells. A recent discovery for editing live cells involves nucleic acid-guided nuclease (e.g., RNA-guided nuclease) editing. A nucleic acid-guided nuclease complexed with an appropriate synthetic guide nucleic acid in a cell can cut the genome of the cell at a desired location. The guide nucleic acid helps the nucleic acid-guided nuclease recognize and cut the DNA at a specific target sequence (either a cellular target sequence or a curing target sequence). By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects, the guide nucleic acid may be a single guide nucleic acid that includes both the crRNA and tracrRNA sequences.

In general, a guide nucleic acid (e.g., gRNA) complexes with a compatible nucleic acid-guided nuclease and can then hybridize with a target sequence, thereby directing the nuclease to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA may be encoded by a DNA sequence on a polynucleotide molecule such as a plasmid, linear construct, or the coding sequence may and preferably does reside within an editing cassette and is under the control of an inducible promoter as described below. For additional information regarding “CREATE” editing cassettes, see U.S. Pat. Nos. 9,982,278; 10,266,849; 10,240,167; 10,351,877; 10,364,442; 10,435,715; 10,465,207; 10,669,559; 10,711,284; and U.S. Ser. No. 16/550,092; and 16,773,712, all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In the present methods and compositions, the guide nucleic acids are provided as a sequence to be expressed from a plasmid or vector and comprises both the guide sequence and the scaffold sequence as a single transcript under the control of an inducible promoter. The guide nucleic acids are engineered to target a desired target sequence (either cellular target sequence or curing target sequence) by altering the guide sequence so that the guide sequence is complementary to a desired target sequence, thereby allowing hybridization between the guide sequence and the target sequence. In general, to generate an edit in the target sequence, the gRNA/nuclease complex binds to a target sequence as determined by the guide RNA, and the nuclease recognizes a protospacer adjacent motif (PAM) sequence adjacent to the target sequence. The target sequence can be any polynucleotide endogenous or exogenous to a prokaryotic or eukaryotic cell, or in vitro. For example, the target sequence can be a polynucleotide residing in the nucleus of a eukaryotic cell. A target sequence can be a sequence encoding a gene product (e.g., a protein) or a non-coding sequence (e.g., a regulatory polynucleotide, an intron, a PAM, or “junk” DNA) or a curing target sequence in an editing vector. In the present description, the target sequence for one of the gRNAs, the curing gRNA, is on the editing vector.

The editing guide nucleic acid may be and preferably is part of an editing cassette that encodes the donor nucleic acid that targets a cellular target sequence. Alternatively, the editing guide nucleic acid may not be part of the editing cassette and instead may be encoded on the editing vector backbone. For example, a sequence coding for an editing guide nucleic acid can be assembled or inserted into a vector backbone first, followed by insertion of the donor nucleic acid in, e.g., an editing cassette. In other cases, the donor nucleic acid in, e.g., an editing cassette can be inserted or assembled into a vector backbone first, followed by insertion of the sequence coding for the editing guide nucleic acid. Preferably, the sequence encoding the editing guide nucleic acid and the donor nucleic acid are located together in a rationally-designed editing cassette and are simultaneously inserted or assembled into a vector backbone to create an editing vector. In yet other embodiments, the sequence encoding the guide nucleic acid and the sequence encoding the donor nucleic acid are both included in the editing cassette.

The target sequence—both the cellular target sequence and the curing target sequence—is associated with a proto-spacer mutation (PAM), which is a short nucleotide sequence recognized by the gRNA/nuclease complex. The precise preferred PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease.

In certain embodiments, the genome editing of a cellular target sequence both introduces a desired DNA change to a cellular target sequence, e.g., the genomic DNA of a cell, and removes, mutates, or renders inactive a proto-spacer mutation (PAM) region in the cellular target sequence. Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

The range of target sequences (both cellular target sequences and curing target sequences) that nucleic acid-guided nucleases can recognize is constrained by the need for a specific PAM to be located near the desired target sequence. As a result, it often can be difficult to target edits with the precision that is necessary for genome editing. It has been found that nucleases can recognize some PAMs very well (e.g., canonical PAMs), and other PAMs less well or poorly (e.g., non-canonical PAMs). Because the methods disclosed herein allow for identification of edited cells in a background of unedited cells, the methods allow for identification of edited cells where the PAM is less than optimal; that is, the methods for identifying edited cells herein allow for identification of edited cells even if editing efficiency is very low. Additionally, the present methods expand the scope of target sequences that may be edited since edits are more readily identified, including cells where the genome edits are associated with less functional PAMs.

As for the nuclease component of the nucleic acid-guided nuclease editing system, a polynucleotide sequence encoding the nucleic acid-guided nuclease can be codon optimized for expression in particular cell types, such as archaeal, prokaryotic or eukaryotic cells. Eukaryotic cells can be yeast, fungi, algae, plant, animal, or human cells. Eukaryotic cells may be those of or derived from a particular organism, such as a mammal, including but not limited to human, mouse, rat, rabbit, dog, or non-human mammals including non-human primates. The choice of nucleic acid-guided nuclease to be employed depends on many factors, such as what type of edit is to be made in the target sequence and whether an appropriate PAM is located close to the desired target sequence. Nucleases of use in the methods described herein include but are not limited to Cas 9, Cas 12/CpfI, MAD2, or MAD7 or other MADzymes and nuclease fusions thereof. Nuclease fusion enzymes typically comprise a CRISPR nucleic acid-guided nuclease engineered to cut one DNA strand in the target DNA rather than making a double-stranded cut, and the nuclease portion is fused to a reverse transcriptase. For more information on nickases and nuclease fusion editing see U.S. Ser. No. 16/740,418; 16/740,420 and 16/740,421, all filed 11 Jan. 2020. As with the guide nucleic acid, the nuclease is encoded by a DNA sequence on a vector (e.g., the engine vector) and be under the control of an inducible promoter. In some embodiments, the inducible promoter may be separate from but the same as the inducible promoter controlling transcription of the guide nucleic acid; that is, a separate inducible promoter drives the transcription of the nuclease or nuclease fusion and guide nucleic acid sequences but the two inducible promoters may be the same type of inducible promoter (e.g., both are pL promoters). Alternatively, the inducible promoter controlling expression of the nuclease may be different from the inducible promoter controlling transcription of the guide nucleic acid; that is, e.g., the nuclease may be under the control of the pBAD inducible promoter, and the guide nucleic acid may be under the control of the pL inducible promoter.

Another component of the nucleic acid-guided nuclease system is the donor nucleic acid comprising homology to the cellular target sequence. In some embodiments, the donor nucleic acid is on the same polynucleotide (e.g., editing vector or editing cassette) as the guide nucleic acid and preferably is (but not necessarily is) under the control of the same promoter as the editing gRNA (e.g., a single promoter driving the transcription of both the editing gRNA and the donor nucleic acid). The donor nucleic acid is designed to serve as a template for homologous recombination with a cellular target sequence nicked or cleaved by the nucleic acid-guided nuclease as a part of the gRNA/nuclease complex. A donor nucleic acid polynucleotide may be of any suitable length, such as about or more than about 20, 25, 50, 75, 100, 150, 200, 500, or 1000 nucleotides in length. In certain preferred aspects, the donor nucleic acid can be provided as an oligonucleotide of between 20-300 nucleotides, more preferably between 50-250 nucleotides. The donor nucleic acid comprises a region that is complementary to a portion of the cellular target sequence (e.g., a homology arm). When optimally aligned, the donor nucleic acid overlaps with (is complementary to) the cellular target sequence by, e.g., about 20, 25, 30, 35, 40, 50, 60, 70, 80, 90 or more nucleotides. The donor nucleic acid comprises two homology arms (regions complementary to the cellular target sequence) flanking the mutation or difference between the donor nucleic acid and the cellular target sequence. The donor nucleic acid comprises at least one mutation or alteration compared to the cellular target sequence, such as an insertion, deletion, modification, or any combination thereof compared to the cellular target sequence.

Again, the donor nucleic acid is preferably provided as part of a rationally-designed editing cassette, which is inserted into an editing vector backbone where the editing vector backbone may comprise a promoter driving transcription of the editing gRNA and the donor DNA, and also comprise a selectable marker different from the selectable marker contained on the engine vector, as well as a curing target sequence that is cut or cleaved during curing. Moreover, there may be more than one, e.g., two, three, four, or more editing gRNA/donor nucleic acid rationally-designed editing cassettes inserted into an editing vector (alternatively, a single rationally-designed editing cassette may comprise two to several editing gRNA/donor DNA pairs), where each editing gRNA is under the control of separate different promoters, separate like promoters, or where all gRNAs/donor nucleic acid pairs are under the control of a single promoter. In preferred embodiments the promoter driving transcription of the editing gRNA and the donor nucleic acid (or driving more than one editing gRNA/donor nucleic acid pair) is an inducible promoter and the promoter driving transcription of the nuclease or nuclease fusion is an inducible promoter as well. In some embodiments and preferably, the nuclease and editing gRNA/donor DNA are under the control of the same inducible promoter.

Inducible editing is advantageous in that singulated cells can be grown for several to many cell doublings before editing is initiated, which increases the likelihood that cells with edits will survive, as the double-strand cuts caused by active editing are largely toxic to the cells. This toxicity results both in cell death in the edited colonies, as well as possibly a lag in growth for the edited cells that do survive but must repair and recover following editing. However, once the edited cells have a chance to recover, the size of the colonies of the edited cells will eventually catch up to the size of the colonies of unedited cells. It is this toxicity, however, that is exploited herein to perform curing.

In addition to the donor nucleic acid, an editing cassette may comprise and preferably does comprise one or more primer sites. The primer sites can be used to amplify the editing cassette by using oligonucleotide primers; for example, if the primer sites flank one or more of the other components of the editing cassette.

Also, as described above, the donor nucleic acid may comprise—in addition to the at least one mutation relative to a cellular target sequence—one or more PAM sequence alterations that mutate, delete or render inactive the PAM site in the cellular target sequence. The PAM sequence alteration in the cellular target sequence renders the PAM site “immune” to the nucleic acid-guided nuclease and protects the cellular target sequence from further editing in subsequent rounds of editing if the same nuclease is used.

In addition, the editing cassette may comprise a barcode. A barcode is a unique DNA sequence that corresponds to the donor DNA sequence such that the barcode can identify the edit made to the corresponding cellular target sequence. The barcode typically comprises four or more nucleotides. In some embodiments, the editing cassettes comprise a collection or library editing gRNAs and of donor nucleic acids representing, e.g., gene-wide or genome-wide libraries of editing gRNAs and donor nucleic acids. The library of editing cassettes is cloned into vector backbones where, e.g., each different donor nucleic acid is associated with a different barcode.

Additionally, in some embodiments, an expression vector or cassette encoding components of the nucleic acid-guided nuclease system further encodes a nucleic acid-guided nuclease comprising one or more nuclear localization sequences (NLSs), such as about or more than about 1, 2, 3, 4, 5, 6, 7, 8, 9, 10, or more NLSs. In some embodiments, the engineered nuclease comprises NLSs at or near the amino-terminus, NLSs at or near the carboxy-terminus, or a combination.

The engine and editing vectors comprise control sequences operably linked to the component sequences to be transcribed. As stated above, the promoters driving transcription of one or more components of the nucleic acid-guided nuclease editing system preferably are inducible. A number of gene regulation control systems have been developed for the controlled expression of genes in plant, microbe, and animal cells, including mammalian cells, including the pL promoter (induced by heat inactivation of the cI857 repressor), the pPhIF promoter (induced by the addition of 2,4 diacetylphloroglucinol (DAPG)), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, Calif.); Bujard and Gossen, PNAS, 89(12):5547-5551 (1992)), the Lac Switch Inducible system (Wyborski et al., Environ Mol Mutagen, 28(4):447-58 (1996); DuCoeur et al., Strategies 5(3):70-72 (1992); U.S. Pat. No. 4,833,080), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others. In the present methods used in the modules and instruments described herein, it is preferred that at least one of the nucleic acid-guided nuclease editing components (e.g., the nuclease and/or the gRNA) is under the control of a promoter that is activated by a rise in temperature, as such a promoter allows for the promoter to be activated by an increase in temperature, and de-activated by a decrease in temperature, thereby “turning off” the editing process. Thus, in the scenario of a promoter that is de-activated by a decrease in temperature, editing in the cell can be turned off without having to change media; to remove, e.g., an inducible biochemical in the medium that is used to induce editing.

### Engineered Microbial Population Dynamics for Biosensing and Information Processing

The present invention leverages high throughput methods of engineering populations of microbes to be resistant or tolerant to a wide variety of small molecule compounds. The disclosure demonstrates that sets of populations of E. coli can be raised to be tolerant and sensitive to furfural, vanillin, 5-methyl-hydroxyfurfural, syringic acid, coumaric acid, resveratrol, and isobutyric acid, among others. Many more growth-inhibiting compounds are well described that have an impact on growth in E. coli, and, indeed, the strain development approach by the high-throughput editing of cells described herein is agnostic to the probe chemical. Thus, a wide variety of selective growth inhibited populations can be developed to selectively enrich a population out of a set of other populations containing other sets of information.

In a first step, a library of random- or rationally-designed editing cassettes is synthesized. Methods and compositions particularly favored for designing and synthesizing editing cassettes such as the exemplary editing vector illustrated on, see U.S. Pat. Nos. 9,982,278; 10,266,849; 10,240,167; 10,351,877; 10,364,442; 10,435,715; 10,465,207; 10,669,559; 10,711,284; 10,731,180 and U.S. Ser. No. 16/550,092; and Ser. No. 16/938,739, all of which are incorporated by reference herein.

Once designed and synthesized, the editing cassettes are amplified, purified and assembled into a vector backbone 104 to create editing cassettes. A number of methods may be used to assemble the editing cassettes including Gibson Assembly®, CPEC, SLIC, Ligase Cycling etc. Additional assembly methods include gap repair in yeast (Bessa, Yeast, 29(10):419-23 (2012)), gateway cloning (Ohtsuka, Curr Pharm Biotechnol, 10(2):244-51 (2009); U.S. Pat. No. 5,888,732 to Hartley et al.; U.S. Pat. No. 6,277,608 to Hartley et al.; and topoisomerase-mediated cloning (Udo, PLoS One, 10(9):e0139349 (2015)); U.S. Pat. No. 6,916,632 B2 to Chestnut et al. These and other nucleic acid assembly techniques are described, e.g., in Sands and Brent, Curr Protoc Mol Biol., 113:3.26.1-3.26.20 (2016); Casini et al., Nat Rev Mol Cell Biol., (9):568-76 (2015); and Patron, Curr Opinion Plant Biol., 19:14-9 (2014)).

In addition to preparing editing cassettes, cells of choice are made electrocompetent for transformation. The cells that can be edited include any prokaryotic, archaeal or eukaryotic cell. For example, prokaryotic cells for use with the present illustrative embodiments can be gram positive bacterial cells, e.g., Bacillus subtilis, or gram-negative bacterial cells, e.g., E. coli cells. Eukaryotic cells for use with the automated multi-module cell editing instruments of the illustrative embodiments include any plant cells and any animal cells, e.g. fungal cells, insect cells, amphibian cells nematode cells, or mammalian cells.

Once the cells of choice are rendered electrocompetent, the cells and editing vectors are combined and the editing vectors are transformed into (e.g., electroporated into) the cells. The cells may be also transformed simultaneously with a separate engine vector expressing an editing nuclease; alternatively and preferably, the cells may already have been transformed with an engine vector configured to express the nuclease; that is, the cells may have already been transformed with an engine vector or the coding sequence for the nuclease may be stably integrated into the cellular genome such that only the editing vector needs to be transformed into the cells.

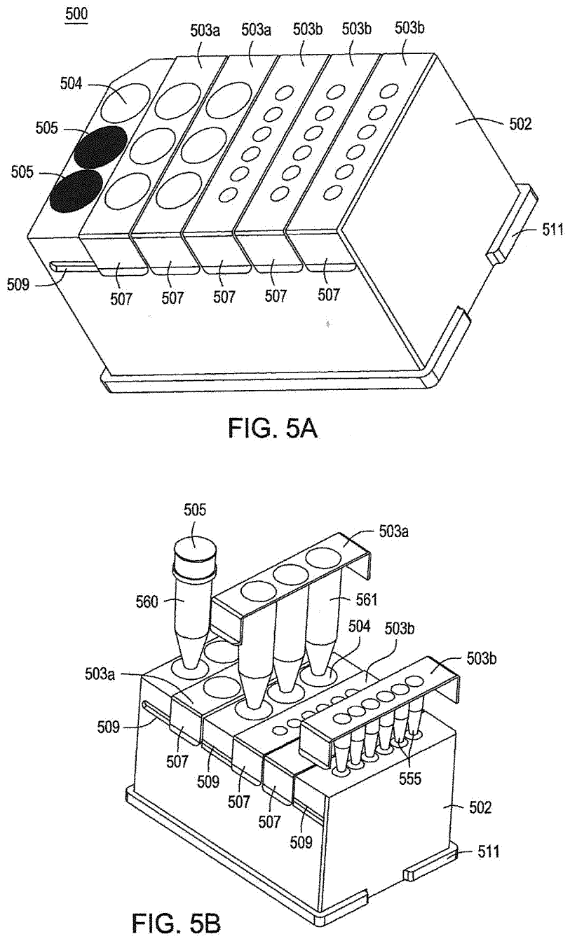

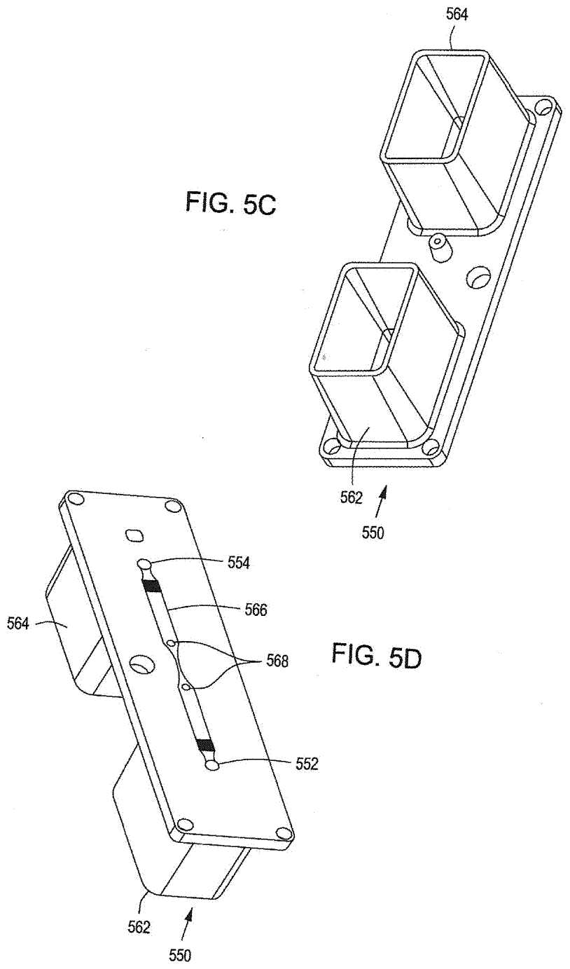

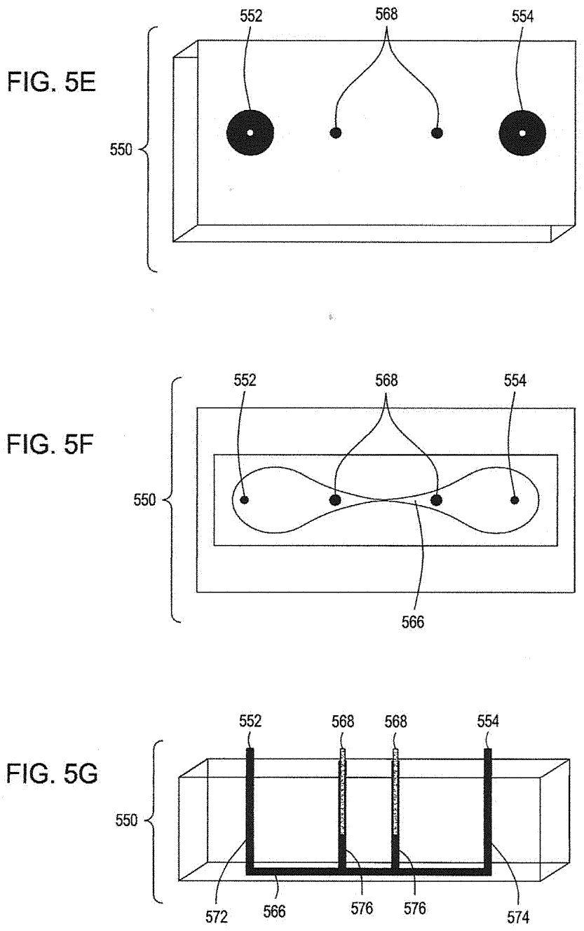

Transformation is intended to include to a variety of art-recognized techniques for introducing an exogenous nucleic acid sequence (e.g., DNA) into a target cell, and the term “transformation” as used herein includes all transformation and transfection techniques. Such methods include, but are not limited to, electroporation, lipofection, optoporation, injection, microprecipitation, microinjection, liposomes, particle bombardment, sonoporation, laser-induced poration, bead transfection, calcium phosphate or calcium chloride co-precipitation, or DEAE-dextran-mediated transfection. Cells can also be prepared for vector uptake using, e.g., a sucrose or glycerol wash. Additionally, hybrid techniques that exploit the capabilities of mechanical and chemical transfection methods can be used, e.g., magnetofection, a transfection methodology that combines chemical transfection with mechanical methods. In another example, cationic lipids may be deployed in combination with gene guns or electroporators. Suitable materials and methods for transforming or transfecting target cells can be found, e.g., in Green and Sambrook, Molecular Cloning: A Laboratory Manual, 4th, ed., Cold Spring Harbor Laboratory Press, Cold Spring Harbor, N.Y., 2014). The present automated methods using the automated multi-module cell processing instrument utilize flow-through electroporation such as the exemplary device shown in FIGS. 5C-5G.

Once transformed, the cells are allowed to recover and selection is performed 108 to select for cells transformed with the editing vector, which in addition to an editing cassette comprises an appropriate selectable marker. As described above, drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 or other selectable markers may be employed.

Following selection for properly transformed cells, editing is induced 110 in the cells by induction of transcription of one or both—preferably both—of the nuclease and gRNA. Induction of transcription of one, or, preferably both, of the nuclease and gRNA is prompted by, e.g., using a pL promoter system where the pL promoter is induced by raising the temperature of the cells in the medium to 42° C. for, e.g., one to many hours to induce expression of the nuclease and gRNA for cutting and editing. A number of gene regulation control systems have been developed for the controlled expression of genes in plant, microbe, and animal cells, including mammalian cells, including, in addition to the pL promoter, the pPhIF promoter (induced by the addition of 2,4 diacetylphloroglucinol (DAPG)), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, Calif.); Bujard and Gossen, PNAS, 89(12):5547-5551 (1992)), the Lac Switch Inducible system (Wyborski et al., Environ Mol Mutagen, 28(4):447-58 (1996); DuCoeur et al., Strategies 5(3):70-72 (1992); U.S. Pat. No. 4,833,080), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others.

The present compositions and methods preferably make use of rationally-designed editing cassettes such as CREATE cassettes, as described above. Each editing cassette comprises an editing gRNA, a donor DNA comprising an intended edit and a PAM or spacer mutation; thus, e.g., a two-cassette multiplex editing cassette comprises a first editing gRNA, a first editing donor DNA, and a first intended edit and a first PAM or spacer mutation, and at least a second editing gRNA, at least a second donor DNA, and at least a second intended edit and a second PAM or spacer mutation. In some embodiments, a single promoter may drive transcription of both the first and second editing gRNAs and both the first and second donor DNAs, and in some embodiments, separate promoters may drive transcription of the first editing gRNA and first donor DNA, and transcription of the second editing gRNA and second donor DNA. In addition, multiplex editing cassettes may comprise nucleic acid elements between the editing cassettes with, e.g., primer sequences, bridging oligonucleotides, and other “cassette-connecting” sequence elements that allow for the assembly of the multiplex editing cassettes.

Once editing is induced, the cells are grown until the cells enter (or are close to entering) the stationary phase of growth, followed by inducing curing of the editing vector by activating an inducible promoter driving transcription of the curing gRNA and inducing the inducible promoter driving transcription of the nuclease. It has been found that curing is particularly effective if the edited cells are in the stationary phase of growth. In yet some aspects, the cells are grown for at least 75% of log phase, 80% of log phase, 85% of log phase, 90% of log phase, 95% of log phase, or are in a stationary phase of growth before inducing curing. Subsequently, a wide variety of selective growth inhibited populations can be developed to selectively enrich a population out of a set of other populations containing other sets of information.

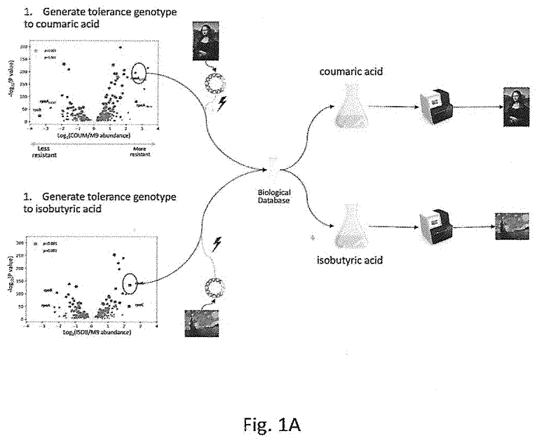

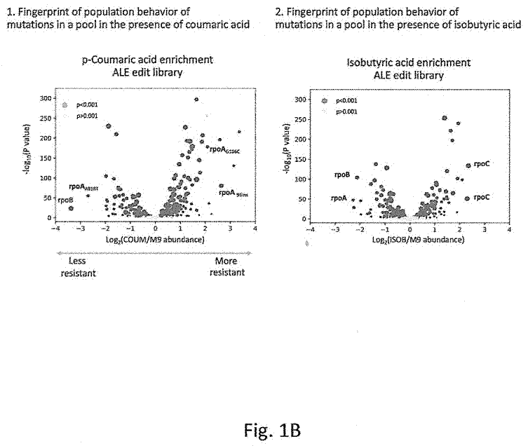

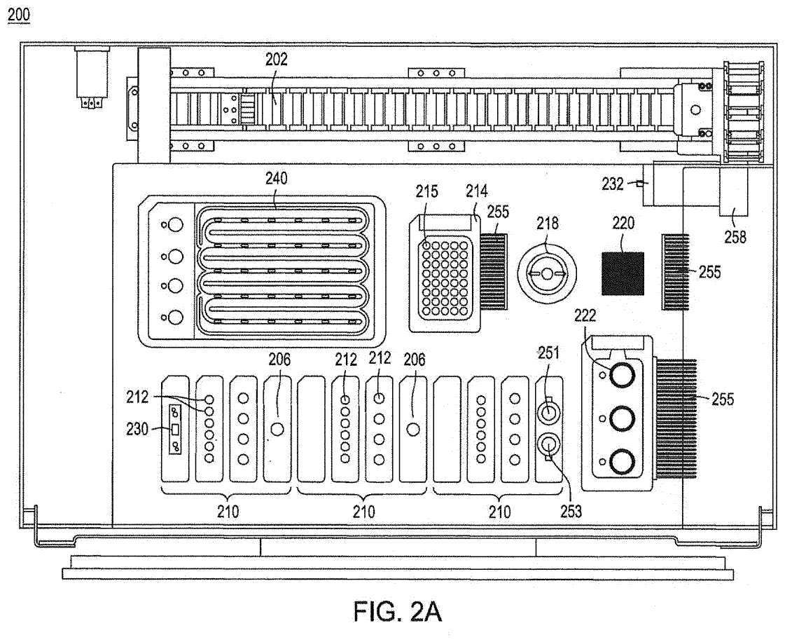

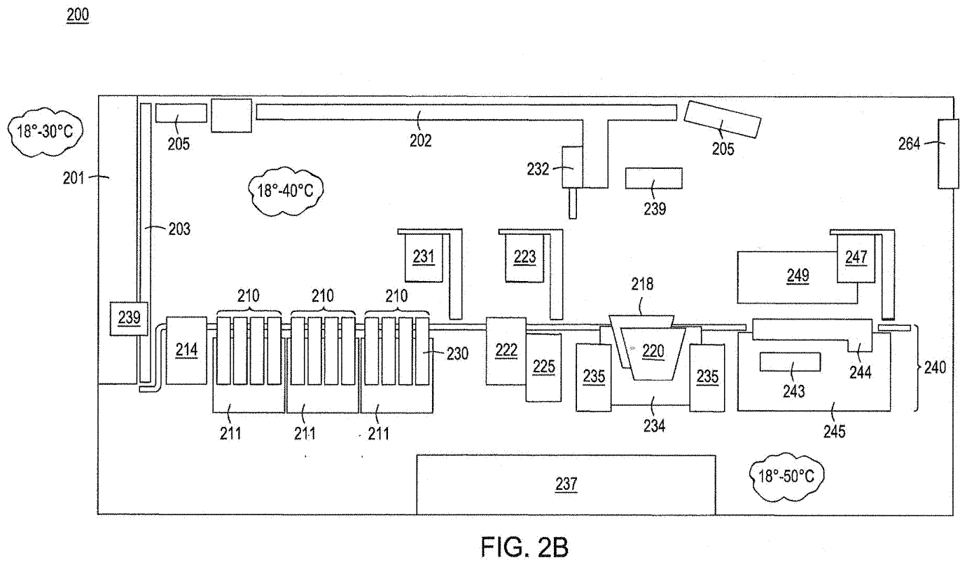

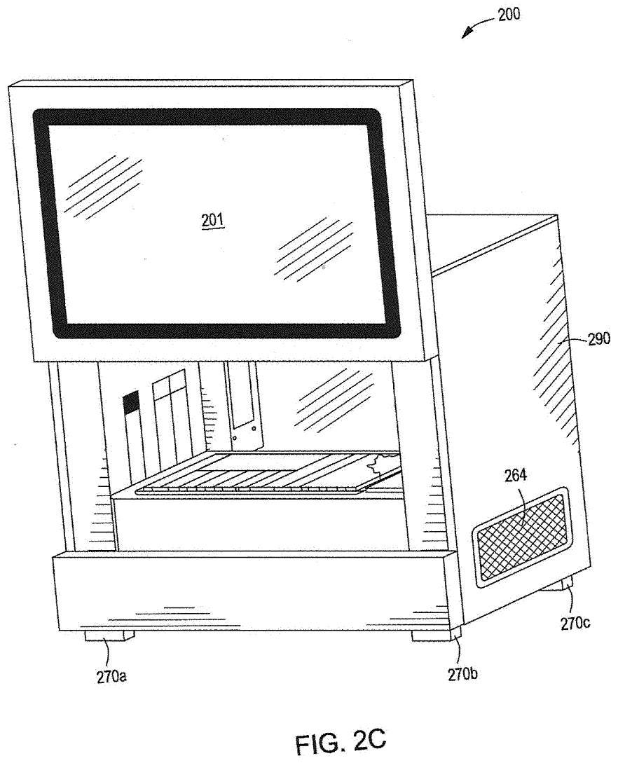

FIG. 2A is a fingerprint of population behavior of mutations in a pool of an exemplary compound, coumaric acid. As shown in FIG. 1A cell populations that were less resistant to coumaric acid tended to have identifiable rpoA and rpoB gene variants (e.g., rpoA_{A818T}). Similarly, cell populations that were more resistant to coumaric acid also tended to have identifiable rpoA and rpoB gene variants, but the variants were different (e.g., rpoA_{G106C} and rpoA₉₆ᵢₙₛ).

FIG. 2B is a fingerprint of population behavior of mutations in a pool of another exemplary compound, isobutyric acid. As shown in FIG. 1B cell populations that were less resistant to isobutyric acid tended to have identifiable rpoA and rpoB gene variants. On the other hand, cell populations that were more resistant to isobutyric acid tended to have identifiable rpoC gene variants.

Additionally, in a further extension of this application, the invention described herein addresses the unmet need for access of information or genetic material from a pool carried by select populations of microbes. Selection of a population of microbes that have been engineered to be tolerant/resistant to a particular chemical can be subsequently extracted from the population of other microbes in the pool. FIG. 1 illustrates this particular embodiment of the disclosure. As shown in FIG. 1, a set of DNA encoding a digital bitmap image with an illustrative content, e.g., an image of Mona Lisa is encoded, and cloned to a group of plasmids. This population of plasmids is then transformed into an exemplary microbial strain, e.g., MG1655 strain containing an insertion of AGAGCG 6-nt insertion at position 96 of rpoA, which as described in FIGS. 2A and 2B provides resistance to coumaric acid and sensitivity to isobutyric acid. A second set of DNA encoding, e.g., an image representation of Starry Night may be encoded, using methods known in the art, and cloned to a group of plasmids. This population of plasmids is that transformed into an MG1655 strain containing a single nucleotide polymorphism mutation in rpoC A104G, which provides growth tolerance to isobutyric acid and sensitivity to coumaric acid. The population can then be mixed together and with other wild-type or engineered tolerance populations to generate a mixed pool containing digital information. Upon needing to extract or otherwise enrich for the digital information of interest, the pool is grown in the presence of the selective compound. The population with tolerance will enrich in the media containing the compound while the other populations will deplete away. After 24 hours of growth, the media is sampled, and DNA is extracted, and the sequences of interest are amplified using a general primer set flanking the region of containing the inserts. The amplification is sequenced and the encoded information is reconstructed from the obtained sequences.

### Automated Cell Editing Instruments and Modules to Perform Nucleic Acid-Guided Nuclease Editing Including Curing

Automated Cell Editing Instruments

FIG. 2A depicts an exemplary automated multi-module cell processing instrument 200 to, e.g., perform one of the exemplary recursive workflows for targeted gene editing of live yeast cells. The instrument 200, for example, may be and preferably is designed as a stand-alone desktop instrument for use within a laboratory environment. The instrument 200 may incorporate a mixture of reusable and disposable components for performing the various integrated processes in conducting automated genome cleavage and/or editing in cells without human intervention. Illustrated is a gantry 202, providing an automated mechanical motion system (actuator) (not shown) that supplies XYZ axis motion control to, e.g., an automated (i.e., robotic) liquid handling system 258 including, e.g., an air displacement pipettor 232 which allows for cell processing among multiple modules without human intervention. In some automated multi-module cell processing instruments, the air displacement pipettor 232 is moved by gantry 202 and the various modules and reagent cartridges remain stationary; however, in other embodiments, the liquid handling system 258 may stay stationary while the various modules and reagent cartridges are moved.

Also included in the automated multi-module cell processing instrument 200 are reagent cartridges 210 comprising reservoirs 212 and transformation module 230 (e.g., a flow-through electroporation device as described in detail in relation to FIGS. 5C-5G and an exemplary reagent cartridge is described in relation to FIGS. 5A and 5B), as well as wash reservoirs 206, cell input reservoir 251 and cell output reservoir 253. The wash reservoirs 206 may be configured to accommodate large tubes, for example, wash solutions, or solutions that are used often throughout an iterative process. Although two of the reagent cartridges 210 comprise a wash reservoir 206 in FIG. 2A, the wash reservoirs instead could be included in a wash cartridge where the reagent and wash cartridges are separate cartridges. In such a case, the reagent cartridge 210 and wash reservoir 206 may be identical except for the consumables (reagents or other components contained within the various inserts) inserted therein.

In some implementations, the reagent cartridges 210 are disposable kits comprising reagents and cells for use in the automated multi-module cell processing/editing instrument 200. For example, a user may open and position each of the reagent cartridges 210 comprising various desired inserts and reagents within the chassis of the automated multi-module cell editing instrument 200 prior to activating cell processing. Further, each of the reagent cartridges 210 may be inserted into receptacles in the chassis having different temperature zones appropriate for the reagents contained therein.

Also illustrated in FIG. 2A is the robotic liquid handling system 258 including the gantry 202 and air displacement pipettor 232. In some examples, the robotic handling system 258 may include an automated liquid handling system such as those manufactured by Tecan Group Ltd. of Mannedorf, Switzerland, Hamilton Company of Reno, Nev. (see, e.g., WO2018015544A1), or Beckman Coulter, Inc. of Fort Collins, Colo. (see, e.g., US20160018427A1). Pipette tips 215 may be provided in a pipette transfer tip supply 214 for use with the air displacement pipettor 232.

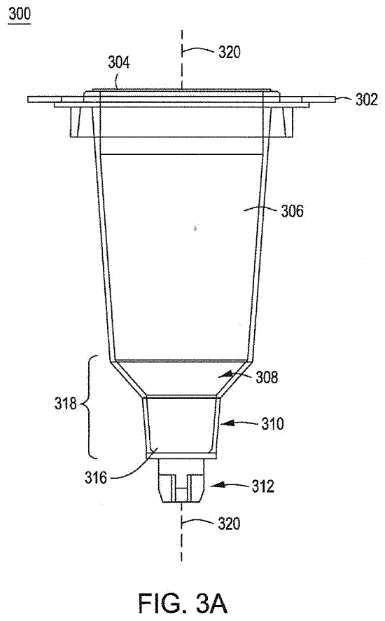

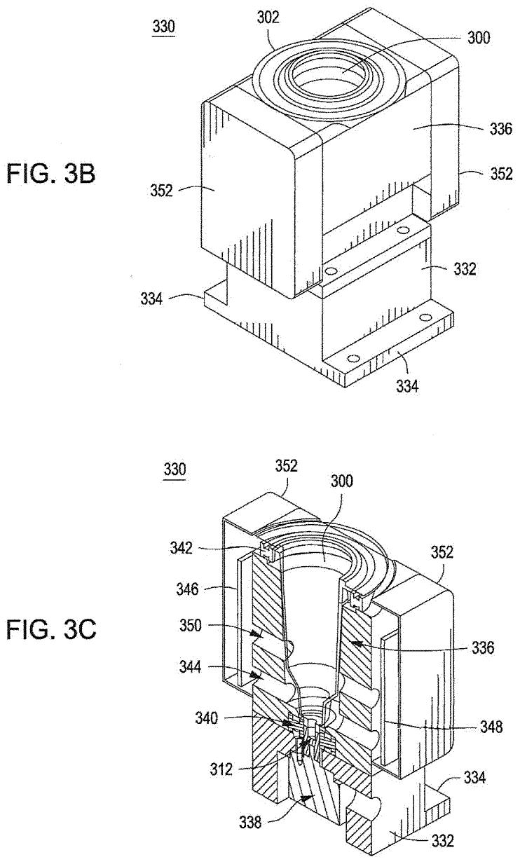

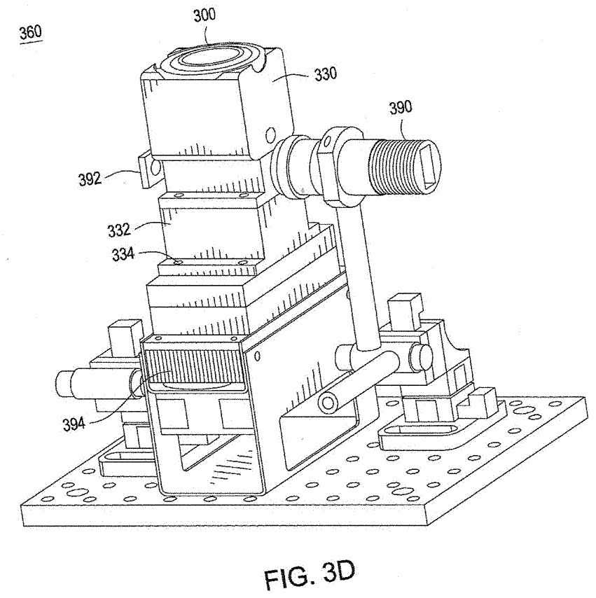

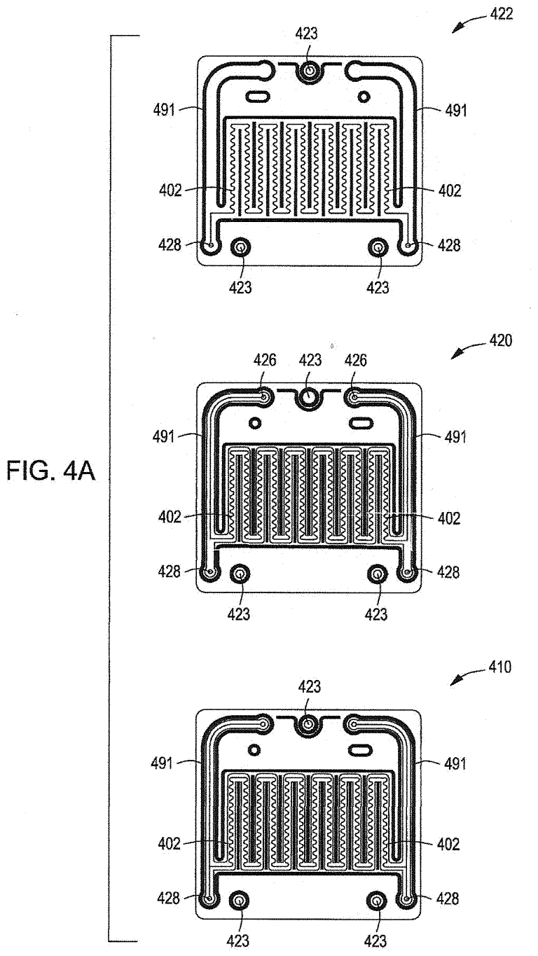

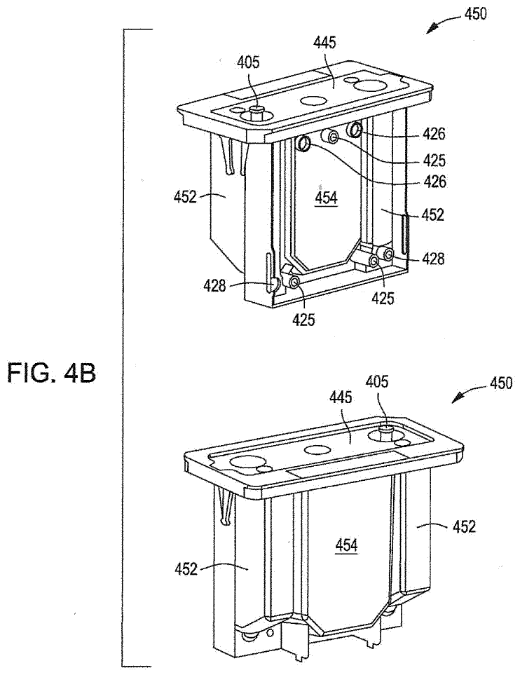

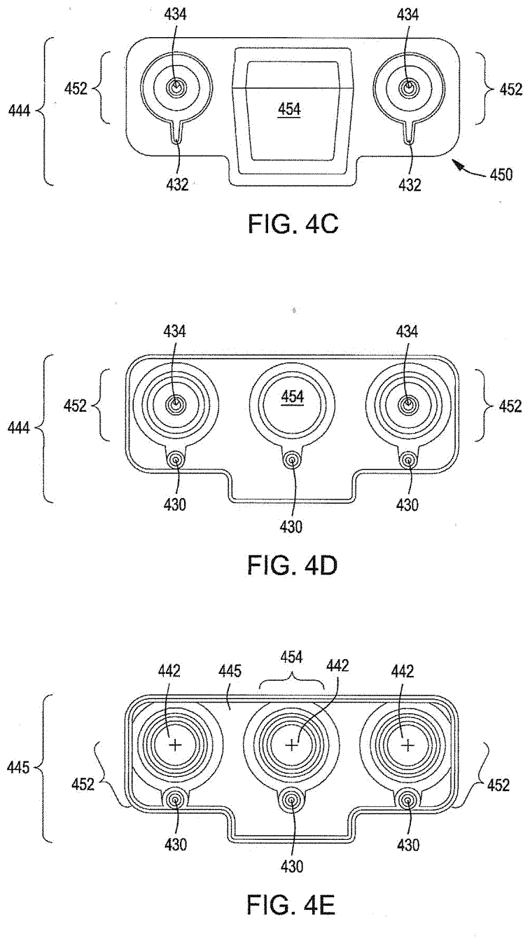

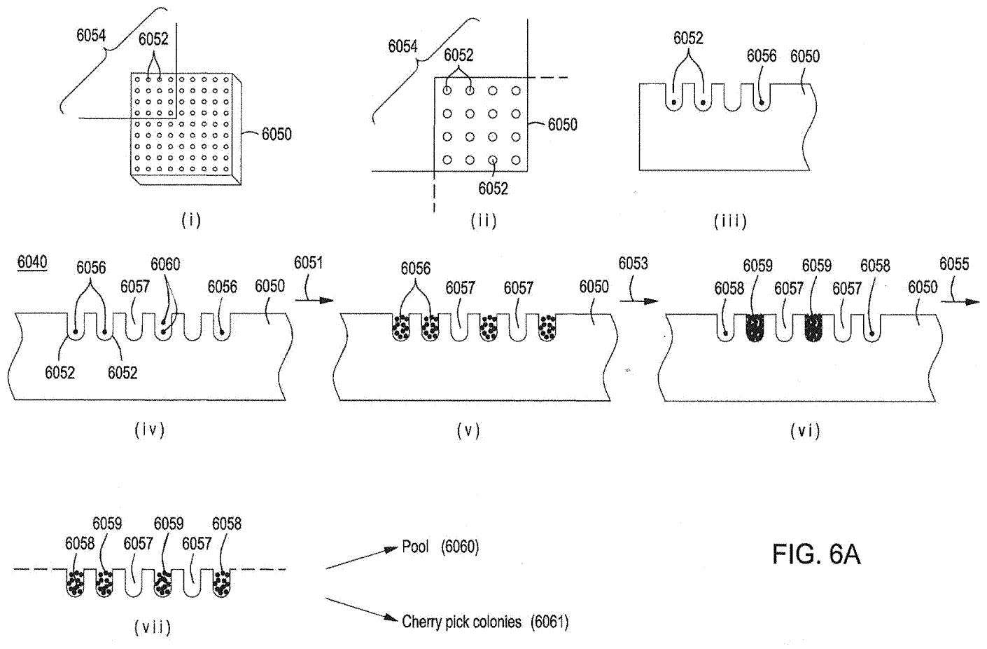

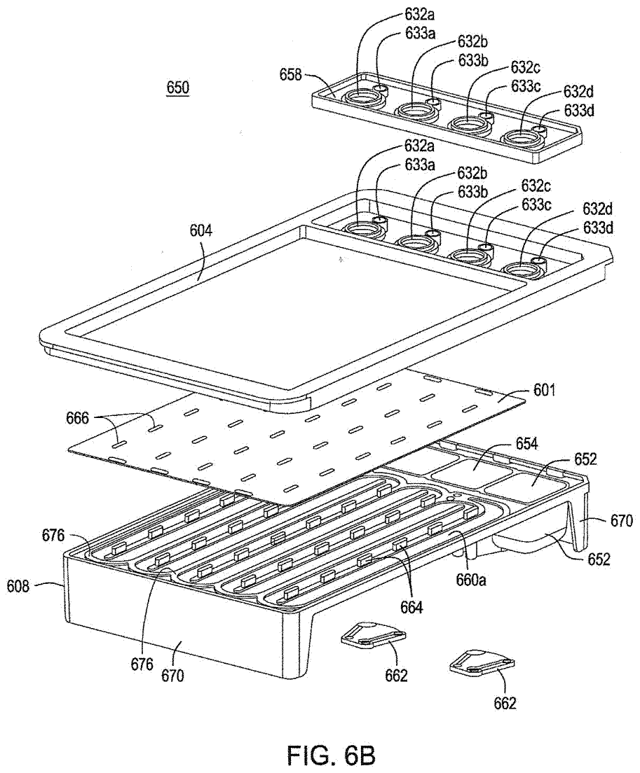

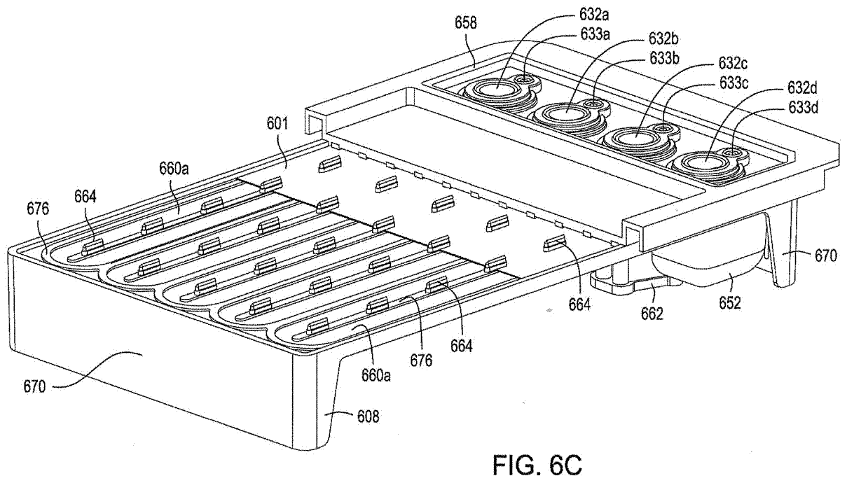

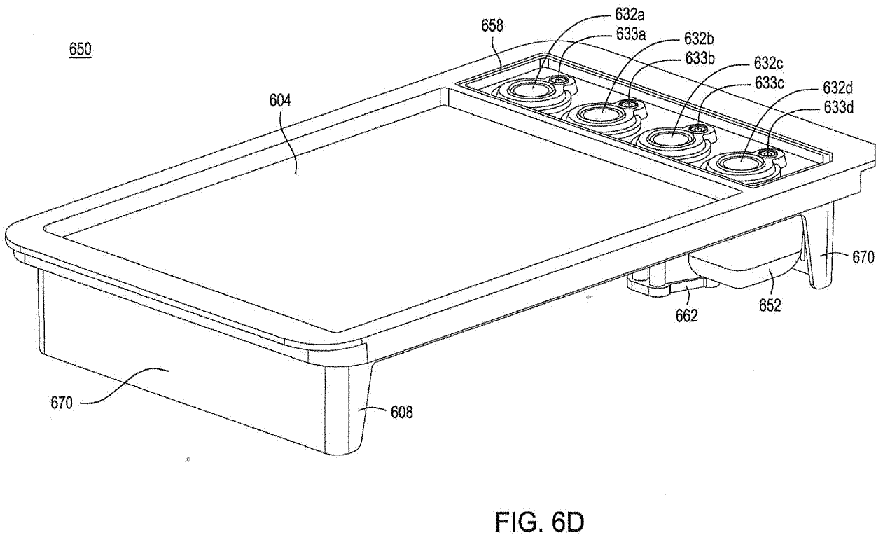

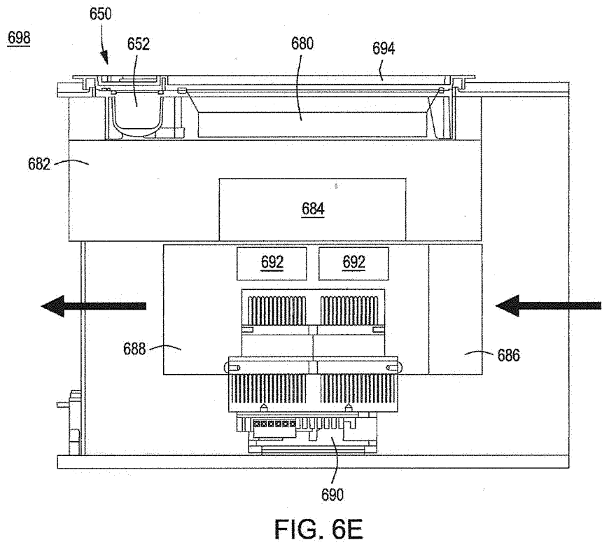

Inserts or components of the reagent cartridges 210, in some implementations, are marked with machine-readable indicia (not shown), such as bar codes, for recognition by the robotic handling system 258. For example, the robotic liquid handling system 258 may scan one or more inserts within each of the reagent cartridges 210 to confirm contents. In other implementations, machine-readable indicia may be marked upon each reagent cartridge 210, and a processing system (not shown, but see element 237 of FIG. 2B) of the automated multi-module cell editing instrument 200 may identify a stored materials map based upon the machine-readable indicia. In the embodiment illustrated in FIG. 2A, a cell growth module comprises a cell growth vial 218 (described in greater detail below in relation to FIGS. 3A-3D). Additionally seen is the TFF module 222 (described above in detail in relation to FIGS. 4A-4E). Also illustrated as part of the automated multi-module cell processing instrument 200 of FIG. 2A is a singulation module 240 (e.g., a solid wall isolation, incubation and normalization device (SWIIN device) is shown here) described herein in relation to FIGS. 6B-6E, served by, e.g., robotic liquid handing system 258 and air displacement pipettor 232. Additionally seen is a selection module 220. Also note the placement of three heatsinks 255.

FIG. 2B is a simplified representation of the contents of the exemplary multi-module cell processing instrument 200 depicted in FIG. 2A. Cartridge-based source materials (such as in reagent cartridges 210), for example, may be positioned in designated areas on a deck of the instrument 200 for access by an air displacement pipettor 232 moved by gantry 202 with pipette tips supplied by pipette transfer tip supply 214. The deck of the multi-module cell processing instrument 200 may include a protection sink such that contaminants spilling, dripping, or overflowing from any of the modules of the instrument 200 are contained within a lip of the protection sink. Also seen are reagent cartridges 210, which are shown disposed with thermal assemblies 211 which can create temperature zones appropriate for different regions. Note that one of the reagent cartridges also comprises a flow-through electroporation device 230 (FTEP), served by FTEP interface (e.g., manifold arm) and actuator 231. Also seen is TFF module 222 with adjacent thermal assembly 225, where the TFF module is served by TFF interface (e.g., manifold arm) and actuator 233. Thermal assemblies 225, 235, and 245 encompass thermal electric devices such as Peltier devices, as well as heatsinks, fans and coolers. The rotating growth vial 218 is within a growth module 234, where the growth module is served by two thermal assemblies 235. Selection module is seen at 220. Also seen is the SWIIN module 240, comprising a SWIIN cartridge (not shown), where the SWIIN module also comprises a thermal assembly 245, illumination 243 (in this embodiment, backlighting), evaporation and condensation control 249, and where the SWIIN module is served by SWIIN interface (e.g., manifold arm) and actuator 247. Also seen in this view is touch screen display 201, display actuator 203, illumination 205 (one on either side of multi-module cell processing instrument 200), cooling grate 264, and cameras 239 (one illumination device on either side of multi-module cell processing instrument 200). Finally, element 237 comprises electronics, such as circuit control boards, high-voltage amplifiers, power supplies, and power entry; as well as pneumatics, such as pumps, valves and sensors.

FIG. 2C illustrates a front perspective view of multi-module cell processing instrument 200 for use in as a desktop version of the automated multi-module cell editing instrument 200. For example, a chassis 290 may have a width of about 24-48 inches, a height of about 24-48 inches and a depth of about 24-48 inches. Chassis 290 may be and preferably is designed to hold all modules and disposable supplies used in automated cell processing and to perform all processes required without human intervention; that is, chassis 290 is configured to provide an integrated, stand-alone automated multi-module cell processing instrument. As illustrated in FIG. 2C, chassis 290 includes touch screen display 201, cooling grate 264, which allows for air flow via an internal fan (not shown). The touch screen display provides information to a user regarding the processing status of the automated multi-module cell editing instrument 200 and accepts inputs from the user for conducting the cell processing. In this embodiment, the chassis 290 is lifted by adjustable feet 270a, 270b, 270c and 270d (feet 270a-270c are shown in this FIG. 2C). Adjustable feet 270a-270d, for example, allow for additional air flow beneath the chassis 290.

Inside the chassis 290, in some implementations, will be most or all of the components described in relation to FIGS. 2A and 2B, including the robotic liquid handling system disposed along a gantry, reagent cartridges 210 including a flow-through electroporation device (not shown in this FIG. 2C), a rotating growth vial 218 in a cell growth module 234 (not shown in this FIG. 2C), a tangential flow filtration module 222 (not shown in this FIG. 2C), a SWIIN module 240 as well as interfaces and actuators for the various modules (not shown in this FIG. 2C). In addition, chassis 290 houses control circuitry, liquid handling tubes, air pump controls, valves, sensors, thermal assemblies (e.g., heating and cooling units) and other control mechanisms (not shown in this FIG. 2C). For examples of multi-module cell editing instruments, see U.S. Pat. Nos. 10,253,316; 10,329,559; 10,323,242; 10,421,959; 10,465,185; 10,519,437; 10,584,333; and 10,584,334 and U.S. Ser. No. 16/750,369, filed 23 Jan. 2020; Ser. No. 16/822,249, filed 18 Mar. 2020; and Ser. No. 16/837,985, filed 1 Apr. 2020, all of which are herein incorporated by reference in their entirety.

The Rotating Cell Growth Module

FIG. 3A shows one embodiment of a rotating growth vial 300 for use with the cell growth device and in the automated multi-module cell processing instruments described herein. The rotating growth vial 300 is an optically-transparent container having an open end 304 for receiving liquid media and cells, a central vial region 306 that defines the primary container for growing cells, a tapered-to-constricted region 318 defining at least one light path 310, a closed end 316, and a drive engagement mechanism 312. The rotating growth vial 300 has a central longitudinal axis 320 around which the vial rotates, and the light path 310 is generally perpendicular to the longitudinal axis of the vial. The first light path 310 is positioned in the lower constricted portion of the tapered-to-constricted region 318. Optionally, some embodiments of the rotating growth vial 300 have a second light path 308 in the tapered region of the tapered-to-constricted region 318. Both light paths in this embodiment are positioned in a region of the rotating growth vial that is constantly filled with the cell culture (cells+growth media) and are not affected by the rotational speed of the growth vial. The first light path 310 is shorter than the second light path 308 allowing for sensitive measurement of OD values when the OD values of the cell culture in the vial are at a high level (e.g., later in the cell growth process), whereas the second light path 308 allows for sensitive measurement of OD values when the OD values of the cell culture in the vial are at a lower level (e.g., earlier in the cell growth process).

The drive engagement mechanism 312 engages with a motor (not shown) to rotate the vial. In some embodiments, the motor drives the drive engagement mechanism 312 such that the rotating growth vial 300 is rotated in one direction only, and in other embodiments, the rotating growth vial 300 is rotated in a first direction for a first amount of time or periodicity, rotated in a second direction (i.e., the opposite direction) for a second amount of time or periodicity, and this process may be repeated so that the rotating growth vial 300 (and the cell culture contents) are subjected to an oscillating motion. Further, the choice of whether the culture is subjected to oscillation and the periodicity therefor may be selected by the user. The first amount of time and the second amount of time may be the same or may be different. The amount of time may be 1, 2, 3, 4, 5, or more seconds, or may be 1, 2, 3, 4 or more minutes. In another embodiment, in an early stage of cell growth the rotating growth vial 400 may be oscillated at a first periodicity (e.g., every 60 seconds), and then a later stage of cell growth the rotating growth vial 300 may be oscillated at a second periodicity (e.g., every one second) different from the first periodicity.

The rotating growth vial 300 may be reusable or, preferably, the rotating growth vial is consumable. In some embodiments, the rotating growth vial is consumable and is presented to the user pre-filled with growth medium, where the vial is hermetically sealed at the open end 304 with a foil seal. A medium-filled rotating growth vial packaged in such a manner may be part of a kit for use with a stand-alone cell growth device or with a cell growth module that is part of an automated multi-module cell processing system. To introduce cells into the vial, a user need only pipette up a desired volume of cells and use the pipette tip to punch through the foil seal of the vial. Open end 304 may optionally include an extended lip 302 to overlap and engage with the cell growth device. In automated systems, the rotating growth vial 300 may be tagged with a barcode or other identifying means that can be read by a scanner or camera (not shown) that is part of the automated system.

The volume of the rotating growth vial 300 and the volume of the cell culture (including growth medium) may vary greatly, but the volume of the rotating growth vial 300 must be large enough to generate a specified total number of cells. In practice, the volume of the rotating growth vial 300 may range from 1-250 mL, 2-100 mL, from 5-80 mL, 10-50 mL, or from 12-35 mL. Likewise, the volume of the cell culture (cells+growth media) should be appropriate to allow proper aeration and mixing in the rotating growth vial 300. Proper aeration promotes uniform cellular respiration within the growth media. Thus, the volume of the cell culture should be approximately 5-85% of the volume of the growth vial or from 20-60% of the volume of the growth vial. For example, for a 30 mL growth vial, the volume of the cell culture would be from about 1.5 mL to about 26 mL, or from 6 mL to about 18 mL.

The rotating growth vial 300 preferably is fabricated from a bio-compatible optically transparent material—or at least the portion of the vial comprising the light path(s) is transparent. Additionally, material from which the rotating growth vial is fabricated should be able to be cooled to about 4° C. or lower and heated to about 55° C. or higher to accommodate both temperature-based cell assays and long-term storage at low temperatures. Further, the material that is used to fabricate the vial must be able to withstand temperatures up to 55° C. without deformation while spinning. Suitable materials include cyclic olefin copolymer (COC), glass, polyvinyl chloride, polyethylene, polyamide, polypropylene, polycarbonate, poly(methyl methacrylate (PMMA), polysulfone, polyurethane, and co-polymers of these and other polymers. Preferred materials include polypropylene, polycarbonate, or polystyrene. In some embodiments, the rotating growth vial is inexpensively fabricated by, e.g., injection molding or extrusion.

FIG. 3B is a perspective view of one embodiment of a cell growth device 330. FIG. 3C depicts a cut-away view of the cell growth device 330 from FIG. 3B. In both figures, the rotating growth vial 300 is seen positioned inside a main housing 336 with the extended lip 302 of the rotating growth vial 300 extending above the main housing 336. Additionally, end housings 352, a lower housing 332 and flanges 334 are indicated in both figures. Flanges 334 are used to attach the cell growth device 330 to heating/cooling means or other structure (not shown). FIG. 3C depicts additional detail. In FIG. 3C, upper bearing 342 and lower bearing 340 are shown positioned within main housing 336. Upper bearing 342 and lower bearing 340 support the vertical load of rotating growth vial 300. Lower housing 332 contains the drive motor 338. The cell growth device 330 of FIG. 3C comprises two light paths: a primary light path 344, and a secondary light path 350. Light path 344 corresponds to light path 310 positioned in the constricted portion of the tapered-to-constricted portion of the rotating growth vial 300, and light path 350 corresponds to light path 308 in the tapered portion of the tapered-to-constricted portion of the rotating growth via 316. Light paths 310 and 308 are not shown in FIG. 3C but may be seen in FIG. 3A. In addition to light paths 344 and 340, there is an emission board 348 to illuminate the light path(s), and detector board 346 to detect the light after the light travels through the cell culture liquid in the rotating growth vial 300.

The motor 338 engages with drive mechanism 312 and is used to rotate the rotating growth vial 300. In some embodiments, motor 338 is a brushless DC type drive motor with built-in drive controls that can be set to hold a constant revolution per minute (RPM) between 0 and about 3000 RPM. Alternatively, other motor types such as a stepper, servo, brushed DC, and the like can be used. Optionally, the motor 338 may also have direction control to allow reversing of the rotational direction, and a tachometer to sense and report actual RPM. The motor is controlled by a processor (not shown) according to, e.g., standard protocols programmed into the processor and/or user input, and the motor may be configured to vary RPM to cause axial precession of the cell culture thereby enhancing mixing, e.g., to prevent cell aggregation, increase aeration, and optimize cellular respiration.

Main housing 336, end housings 352 and lower housing 332 of the cell growth device 330 may be fabricated from any suitable, robust material including aluminum, stainless steel, and other thermally conductive materials, including plastics. These structures or portions thereof can be created through various techniques, e.g., metal fabrication, injection molding, creation of structural layers that are fused, etc. Whereas the rotating growth vial 300 is envisioned in some embodiments to be reusable, but preferably is consumable, the other components of the cell growth device 330 are preferably reusable and function as a stand-alone benchtop device or as a module in a multi-module cell processing system.

The processor (not shown) of the cell growth device 330 may be programmed with information to be used as a “blank” or control for the growing cell culture. A “blank” or control is a vessel containing cell growth medium only, which yields 100% transmittance and 0 OD, while the cell sample will deflect light rays and will have a lower percent transmittance and higher OD. As the cells grow in the media and become denser, transmittance will decrease and OD will increase. The processor (not shown) of the cell growth device 330—may be programmed to use wavelength values for blanks commensurate with the growth media typically used in cell culture (whether, e.g., mammalian cells, bacterial cells, animal cells, yeast cells, etc.). Alternatively, a second spectrophotometer and vessel may be included in the cell growth device 330, where the second spectrophotometer is used to read a blank at designated intervals.

FIG. 3D illustrates a cell growth device 330 as part of an assembly comprising the cell growth device 330 of FIG. 3B coupled to light source 390, detector 392, and thermal components 394. The rotating growth vial 300 is inserted into the cell growth device. Components of the light source 390 and detector 392 (e.g., such as a photodiode with gain control to cover 5-log) are coupled to the main housing of the cell growth device. The lower housing 332 that houses the motor that rotates the rotating growth vial 300 is illustrated, as is one of the flanges 334 that secures the cell growth device 330 to the assembly. Also, the thermal components 394 illustrated are a Peltier device or thermoelectric cooler. In this embodiment, thermal control is accomplished by attachment and electrical integration of the cell growth device 330 to the thermal components 394 via the flange 334 on the base of the lower housing 332. Thermoelectric coolers are capable of “pumping” heat to either side of a junction, either cooling a surface or heating a surface depending on the direction of current flow. In one embodiment, a thermistor is used to measure the temperature of the main housing and then, through a standard electronic proportional-integral-derivative (PID) controller loop, the rotating growth vial 300 is controlled to approximately +/−0.5° C.

In use, cells are inoculated (cells can be pipetted, e.g., from an automated liquid handling system or by a user) into pre-filled growth media of a rotating growth vial 300 by piercing though the foil seal or film. The programmed software of the cell growth device 330 sets the control temperature for growth, typically 30° C., then slowly starts the rotation of the rotating growth vial 300. The cell/growth media mixture slowly moves vertically up the wall due to centrifugal force allowing the rotating growth vial 300 to expose a large surface area of the mixture to a normal oxygen environment. The growth monitoring system takes either continuous readings of the OD or OD measurements at pre-set or pre-programmed time intervals. These measurements are stored in internal memory and if requested the software plots the measurements versus time to display a growth curve. If enhanced mixing is required, e.g., to optimize growth conditions, the speed of the vial rotation can be varied to cause an axial precession of the liquid, and/or a complete directional change can be performed at programmed intervals. The growth monitoring can be programmed to automatically terminate the growth stage at a pre-determined OD, and then quickly cool the mixture to a lower temperature to inhibit further growth.

One application for the cell growth device 330 is to constantly measure the optical density of a growing cell culture. One advantage of the described cell growth device is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 minutes. While the cell growth device 330 has been described in the context of measuring the optical density (OD) of a growing cell culture, it should, however, be understood by a skilled artisan given the teachings of the present specification that other cell growth parameters can be measured in addition to or instead of cell culture OD. As with optional measure of cell growth in relation to the solid wall device or module described supra, spectroscopy using visible, UV, or near infrared (NIR) light allows monitoring the concentration of nutrients and/or wastes in the cell culture and other spectroscopic measurements may be made; that is, other spectral properties can be measured via, e.g., dielectric impedance spectroscopy, visible fluorescence, fluorescence polarization, or luminescence. Additionally, the cell growth device 330 may include additional sensors for measuring, e.g., dissolved oxygen, carbon dioxide, pH, conductivity, and the like. For additional details regarding rotating growth vials and cell growth devices see U.S. Pat. Nos. 10,435,662; 10,443,031; 10,590,375 and U.S. Ser. No. 16/780,640, filed 3 Feb. 2020.

The Cell Concentration Module

As described above in relation to the rotating growth vial and cell growth module, in order to obtain an adequate number of cells for transformation or transfection, cells typically are grown to a specific optical density in medium appropriate for the growth of the cells of interest; however, for effective transformation or transfection, it is desirable to decrease the volume of the cells as well as render the cells competent via buffer or medium exchange. Thus, one sub-component or module that is desired in cell processing systems for the processes listed above is a module or component that can grow, perform buffer exchange, and/or concentrate cells and render them competent so that they may be transformed or transfected with the nucleic acids needed for engineering or editing the cell's genome.

FIG. 4A shows a retentate member 422 (top), permeate member 420 (middle) and a tangential flow assembly 410 (bottom) comprising the retentate member 422, membrane 424 (not seen in FIG. 4A), and permeate member 420 (also not seen). In FIG. 4A, retentate member 422 comprises a tangential flow channel 402, which has a serpentine configuration that initiates at one lower corner of retentate member 422 specifically at retentate port 428—traverses across and up then down and across retentate member 422, ending in the other lower corner of retentate member 422 at a second retentate port 428. Also seen on retentate member 422 are energy directors 491, which circumscribe the region where a membrane or filter (not seen in this FIG. 4A) is seated, as well as interdigitate between areas of channel 402. Energy directors 491 in this embodiment mate with and serve to facilitate ultrasonic welding or bonding of retentate member 422 with permeate/filtrate member 420 via the energy director component 491 on permeate/filtrate member 420 (at right). Additionally, countersinks 423 can be seen, two on the bottom one at the top middle of retentate member 422. Countersinks 423 are used to couple and tangential flow assembly 410 to a reservoir assembly (not seen in this FIG. 4A but see FIG. 4B).

Permeate/filtrate member 420 is seen in the middle of FIG. 4A and comprises, in addition to energy director 491, through-holes for retentate ports 428 at each bottom corner (which mate with the through-holes for retentate ports 428 at the bottom corners of retentate member 422), as well as a tangential flow channel 402 and two permeate/filtrate ports 426 positioned at the top and center of permeate member 420. The tangential flow channel 402 structure in this embodiment has a serpentine configuration and an undulating geometry, although other geometries may be used. Permeate member 420 also comprises countersinks 423, coincident with the countersinks 423 on retentate member 420.

At bottom of FIG. 4A is a tangential flow assembly 410 comprising the retentate member 422 and permeate member 420 seen in this FIG. 4A. In this view, retentate member 422 is “on top” of the view, a membrane (not seen in this view of the assembly) would be adjacent and under retentate member 422 and permeate member 420 (also not seen in this view of the assembly) is adjacent to and beneath the membrane. Again countersinks 423 are seen, where the countersinks in the retentate member 422 and the permeate member 420 are coincident and configured to mate with threads or mating elements for the countersinks disposed on a reservoir assembly (not seen in FIG. 4A but see FIG. 4B).

A membrane or filter is disposed between the retentate and permeate members, where fluids can flow through the membrane but cells cannot and are thus retained in the flow channel disposed in the retentate member. Filters or membranes appropriate for use in the TFF device/module are those that are solvent resistant, are contamination free during filtration, and are able to retain the types and sizes of cells of interest. For example, in order to retain small cell types such as bacterial cells, pore sizes can be as low as 0.2 μm, however for other cell types, the pore sizes can be as high as 20 μm. Indeed, the pore sizes useful in the TFF device/module include filters with sizes from 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, glass fiber, or metal substrates as in the case of laser or electrochemical etching.

The length of the channel structure 402 may vary depending on the type and volume of the cell culture to be grown and the optical density of the cell culture to be concentrated. The length of the channel structure typically is from 60 mm to 300 mm, or from 70 mm to 200 mm, or from 80 mm to 100 mm. The cross-section configuration of the flow channel 402 may be round, elliptical, oval, square, rectangular, trapezoidal, or irregular. If square, rectangular, or another shape with generally straight sides, the cross section may be from about 10 μm to 1000 μm wide, or from 200 μm to 800 μm wide, or from 300 μm to 700 μm wide, or from 400 μm to 600 μm wide; and from about 10 μm to 1000 μm high, or from 200 μm to 800 μm high, or from 300 μm to 700 μm high, or from 400 μm to 600 μm high. If the cross section of the flow channel 402 is generally round, oval or elliptical, the radius of the channel may be from about 50 μm to 1000 μm in hydraulic radius, or from 5 μm to 800 μm in hydraulic radius, or from 200 μm to 700 μm in hydraulic radius, or from 300 μm to 600 μm wide in hydraulic radius, or from about 200 to 500 μm in hydraulic radius. Moreover, the volume of the channel in the retentate 422 and permeate 420 members may be different depending on the depth of the channel in each member.

FIG. 4B shows front perspective (top) and rear perspective (bottom) views of a reservoir assembly 450 configured to be used with the tangential flow assembly 410 seen in FIG. 4A. Seen in the front perspective view (e.g., “front” being the side of reservoir assembly 450 that is coupled to the tangential flow assembly 410 (seen in FIG. 4A) are retentate reservoirs 452 on either side of permeate reservoir 454. Also seen are permeate ports 426, retentate ports 428, and three threads or mating elements 425 for countersinks 423 (countersinks 423 not seen in this FIG. 4B). Threads or mating elements 425 for countersinks 423 are configured to mate or couple the tangential flow assembly 410 (seen in FIG. 4A) to reservoir assembly 450. Alternatively or in addition, fasteners, sonic welding or heat stakes may be used to mate or couple the tangential flow assembly 410 to reservoir assembly 450. In addition is seen gasket 445 covering the top of reservoir assembly 450, with pipette tip 405 shown inserted into the left-most retentate reservoir. Gasket 445 is described in detail in relation to FIG. 4E. At left in FIG. 4B is a rear perspective view of reservoir assembly 450, where “rear” is the side of reservoir assembly 450 that is not coupled to the tangential flow assembly. Seen are retentate reservoirs 452, permeate reservoir 454, gasket 445, with pipette tip 405 shown inserted into the right-most retentate reservoir.

The TFF device may be fabricated from any robust material in which channels (and channel branches) may be milled including stainless steel, silicon, glass, aluminum, or plastics including cyclic-olefin copolymer (COC), cyclo-olefin polymer (COP), polystyrene, polyvinyl chloride, polyethylene, polyamide, polyethylene, polypropylene, acrylonitrile butadiene, polycarbonate, polyetheretheketone (PEEK), poly(methyl methylacrylate) (PMMA), polysulfone, and polyurethane, and co-polymers of these and other polymers. If the TFF device/module is disposable, preferably it is made of plastic. In some embodiments, the material used to fabricate the TFF device/module is thermally-conductive so that the cell culture may be heated or cooled to a desired temperature. In certain embodiments, the TFF device is formed by precision mechanical machining, laser machining, electro discharge machining (for metal devices); wet or dry etching (for silicon devices); dry or wet etching, powder or sandblasting, photostructuring (for glass devices); or thermoforming, injection molding, hot embossing, or laser machining (for plastic devices) using the materials mentioned above that are amenable to this mass production techniques.

FIG. 4C depicts a top-down view of the reservoir assemblies 450 shown in FIG. 4B. FIG. 4D depicts a cover 444 for reservoir assembly 450 shown in FIGS. 4B and 4E depicts a gasket 445 that in operation is disposed on cover 444 of reservoir assemblies 450 shown in FIG. 4B. FIG. 4C is a top-down view of reservoir assembly 450, showing the tops of the two retentate reservoirs 452, one on either side of permeate reservoir 454. Also seen are grooves 432 that will mate with a pneumatic port (not shown), and fluid channels 434 that reside at the bottom of retentate reservoirs 452, which fluidically couple the retentate reservoirs 452 with the retentate ports 428 (not shown), via the through-holes for the retentate ports in permeate member 420 and membrane 424 (also not shown). FIG. 4D depicts a cover 444 that is configured to be disposed upon the top of reservoir assembly 450. Cover 444 has round cut-outs at the top of retentate reservoirs 452 and permeate/filtrate reservoir 454. Again at the bottom of retentate reservoirs 452 fluid channels 434 can be seen, where fluid channels 434 fluidically couple retentate reservoirs 452 with the retentate ports 428 (not shown). Also shown are three pneumatic ports 430 for each retentate reservoir 452 and permeate/filtrate reservoir 454. FIG. 4E depicts a gasket 445 that is configured to be disposed upon the cover 444 of reservoir assembly 450. Seen are three fluid transfer ports 442 for each retentate reservoir 452 and for permeate/filtrate reservoir 454. Again, three pneumatic ports 430, for each retentate reservoir 452 and for permeate/filtrate reservoir 454, are shown.

The overall work flow for cell growth comprises loading a cell culture to be grown into a first retentate reservoir, optionally bubbling air or an appropriate gas through the cell culture, passing or flowing the cell culture through the first retentate port then tangentially through the TFF channel structure while collecting medium or buffer through one or both of the permeate ports 426, collecting the cell culture through a second retentate port 428 into a second retentate reservoir, optionally adding additional or different medium to the cell culture and optionally bubbling air or gas through the cell culture, then repeating the process, all while measuring, e.g., the optical density of the cell culture in the retentate reservoirs continuously or at desired intervals. Measurements of optical densities (OD) at programmed time intervals are accomplished using a 600 nm Light Emitting Diode (LED) that has been columnated through an optic into the retentate reservoir(s) containing the growing cells. The light continues through a collection optic to the detection system which consists of a (digital) gain-controlled silicone photodiode. Generally, optical density is shown as the absolute value of the logarithm with base 10 of the power transmission factors of an optical attenuator: OD=−log10 (Power out/Power in). Since OD is the measure of optical attenuation—that is, the sum of absorption, scattering, and reflection—the TFF device OD measurement records the overall power transmission, so as the cells grow and become denser in population, the OD (the loss of signal) increases. The OD system is pre-calibrated against OD standards with these values stored in an on-board memory accessible by the measurement program.

In the channel structure, the membrane bifurcating the flow channels retains the cells on one side of the membrane (the retentate side 422) and allows unwanted medium or buffer to flow across the membrane into a filtrate or permeate side (e.g., permeate member 420) of the device. Bubbling air or other appropriate gas through the cell culture both aerates and mixes the culture to enhance cell growth. During the process, medium that is removed during the flow through the channel structure is removed through the permeate/filtrate ports 406. Alternatively, cells can be grown in one reservoir with bubbling or agitation without passing the cells through the TFF channel from one reservoir to the other.

The overall work flow for cell concentration using the TFF device/module involves flowing a cell culture or cell sample tangentially through the channel structure. As with the cell growth process, the membrane bifurcating the flow channels retains the cells on one side of the membrane and allows unwanted medium or buffer to flow across the membrane into a permeate/filtrate side (e.g., permeate member 420) of the device. In this process, a fixed volume of cells in medium or buffer is driven through the device until the cell sample is collected into one of the retentate ports 428, and the medium/buffer that has passed through the membrane is collected through one or both of the permeate/filtrate ports 426. All types of prokaryotic and eukaryotic cells—both adherent and non-adherent cells—can be grown in the TFF device. Adherent cells may be grown on beads or other cell scaffolds suspended in medium that flow through the TFF device.

The medium or buffer used to suspend the cells in the cell concentration device/module may be any suitable medium or buffer for the type of cells being transformed or transfected, such as LB, SOC, TPD, YPG, YPAD, MEM, DMEM, IMDM, RPMI, Hanks′, PBS and Ringer's solution, where the media may be provided in a reagent cartridge as part of a kit.

In both the cell growth and concentration processes, passing the cell sample through the TFF device and collecting the cells in one of the retentate ports 404 while collecting the medium in one of the permeate/filtrate ports 406 is considered “one pass” of the cell sample. The transfer between retentate reservoirs “flips” the culture. The retentate and permeate ports collecting the cells and medium, respectively, for a given pass reside on the same end of TFF device/module with fluidic connections arranged so that there are two distinct flow layers for the retentate and permeate/filtrate sides, but if the retentate port 404 resides on the retentate member of device/module (that is, the cells are driven through the channel above the membrane and the filtrate (medium) passes to the portion of the channel below the membrane), the permeate/filtrate port 406 will reside on the permeate member of device/module and vice versa (that is, if the cell sample is driven through the channel below the membrane, the filtrate (medium) passes to the portion of the channel above the membrane). Due to the high pressures used to transfer the cell culture and fluids through the flow channel of the TFF device, the effect of gravity is negligible.

At the conclusion of a “pass” in either of the growth and concentration processes, the cell sample is collected by passing through the retentate port 428 and into the retentate reservoir (not shown). To initiate another “pass”, the cell sample is passed again through the TFF device, this time in a flow direction that is reversed from the first pass. The cell sample is collected by passing through the retentate port 428 and into retentate reservoir (not shown) on the opposite end of the device/module from the retentate port 428 that was used to collect cells during the first pass. Likewise, the medium/buffer that passes through the membrane on the second pass is collected through the permeate port 426 on the opposite end of the device/module from the permeate port 426 that was used to collect the filtrate during the first pass, or through both ports. This alternating process of passing the retentate (the concentrated cell sample) through the device/module is repeated until the cells have been grown to a desired optical density, and/or concentrated to a desired volume, and both permeate ports (i.e., if there are more than one) can be open during the passes to reduce operating time. In addition, buffer exchange may be effected by adding a desired buffer (or fresh medium) to the cell sample in the retentate reservoir, before initiating another “pass”, and repeating this process until the old medium or buffer is diluted and filtered out and the cells reside in fresh medium or buffer. Note that buffer exchange and cell growth may (and typically do) take place simultaneously, and buffer exchange and cell concentration may (and typically do) take place simultaneously. For further information and alternative embodiments on TFFs see, e.g., U.S. Ser. No. 16/798,302, filed 22 Feb. 2020.

Nucleic Acid Assembly Module

Certain embodiments of the automated multi-module cell editing instruments of the present disclosure optionally include a nucleic acid assembly module. The nucleic acid assembly module is configured to accept and assemble the nucleic acids necessary to facilitate the desired genome editing events. In general, the term “vector” refers to a nucleic acid molecule capable of transporting a desired nucleic acid to which it has been linked into a cell. Vectors include, but are not limited to, nucleic acid molecules that are single-stranded, double-stranded, or partially double-stranded; nucleic acid molecules that include one or more free ends, no free ends (e.g., circular); nucleic acid molecules that include DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, where virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors” or “editing vectors.” Common expression vectors of utility in recombinant DNA techniques are often in the form of plasmids. Additional vectors include fosmids, phagemids, and synthetic chromosomes.

Recombinant expression vectors can include a nucleic acid in a form suitable for transcription, and for some nucleic acid sequences, translation and expression of the nucleic acid in a host cell, which means that the recombinant expression vectors include one or more regulatory elements—which may be selected on the basis of the host cells to be used for expression—that are operatively-linked to the nucleic acid sequence to be expressed. Within a recombinant expression vector, “operably linked” is intended to mean that the nucleotide sequence of interest is linked to the regulatory element(s) in a manner that allows for transcription, and for some nucleic acid sequences, translation and expression of the nucleotide sequence (e.g. in an in vitro transcription/translation system or in a host cell when the vector is introduced into the host cell). Appropriate recombination and cloning methods are disclosed in US Pub. No. 2004/0171156, the contents of which are herein incorporated by reference in their entirety for all purposes.

Regulatory elements are operably linked to one or more elements of a targetable nuclease system so as to drive transcription, and for some nucleic acid sequences, translation and expression of the one or more components of the targetable nuclease system.

In addition, the polynucleotide sequence encoding the nucleic acid-guided nuclease can be codon optimized for expression in particular cells, such as prokaryotic or eukaryotic cells. Eukaryotic cells can be yeast, fungi, algae, plant, animal, or human cells. Eukaryotic cells may be those of or derived from a particular organism, such as a mammal, including but not limited to human, mouse, rat, rabbit, dog, or non-human mammal including non-human primate. In addition or alternatively, a vector may include a regulatory element operably liked to a polynucleotide sequence, which, when transcribed, forms a guide RNA.

The nucleic acid assembly module can be configured to perform a wide variety of different nucleic acid assembly techniques in an automated fashion. Nucleic acid assembly techniques that can be performed in the nucleic acid assembly module of the disclosed automated multi-module cell editing instruments include, but are not limited to, those assembly methods that use restriction endonucleases, including PCR, BioBrick assembly (U.S. Pat. No. 9,361,427), Type IIS cloning (e.g., GoldenGate assembly, European Patent Application Publication EP 2 395 087 A1), and Ligase Cycling Reaction (de Kok, ACS Synth Biol., 3(2):97-106 (2014); Engler, et al., PLoS One, 3(11):e3647 (2008); and U.S. Pat. No. 6,143,527). In other embodiments, the nucleic acid assembly techniques performed by the disclosed automated multi-module cell editing instruments are based on overlaps between adjacent parts of the nucleic acids, such as Gibson Assembly®, CPEC, SLIC, Ligase Cycling etc. Additional assembly methods include gap repair in yeast (Bessa, Yeast, 29(10):419-23 (2012)), gateway cloning (Ohtsuka, Curr Pharm Biotechnol, 10(2):244-51 (2009)); U.S. Pat. Nos. 5,888,732; and 6,277,608), and topoisomerase-mediated cloning (Udo, PLoS One, 10(9):e0139349 (2015); and U.S. Pat. No. 6,916,632). These and other nucleic acid assembly techniques are described, e.g., in Sands and Brent, Curr Protoc Mol Biol., 113:3.26.1-3.26.20 (2016).

The nucleic acid assembly module is temperature controlled depending upon the type of nucleic acid assembly used in the automated multi-module cell editing instrument. For example, when PCR is utilized in the nucleic acid assembly module, the module includes a thermocycling capability allowing the temperatures to cycle between denaturation, annealing and extension steps. When single temperature assembly methods (e.g., isothermal assembly methods) are utilized in the nucleic acid assembly module, the module provides the ability to reach and hold at the temperature that optimizes the specific assembly process being performed. These temperatures and the duration for maintaining these temperatures can be determined by a preprogrammed set of parameters executed by a script, or manually controlled by the user using the processing system of the automated multi-module cell editing instrument.

In one embodiment, the nucleic acid assembly module is a module to perform assembly using a single, isothermal reaction. Certain isothermal assembly methods can combine simultaneously up to 15 nucleic acid fragments based on sequence identity. The assembly method provides, in some embodiments, nucleic acids to be assembled which include an approximate 20-40 base overlap with adjacent nucleic acid fragments. The fragments are mixed with a cocktail of three enzymes—an exonuclease, a polymerase, and a ligase-along with buffer components. Because the process is isothermal and can be performed in a 1-step or 2-step method using a single reaction vessel, isothermal assembly reactions are ideal for use in an automated multi-module cell editing instrument. The 1-step method allows for the assembly of up to five different fragments using a single step isothermal process. The fragments and the master mix of enzymes are combined and incubated at 50° C. for up to one hour. For the creation of more complex constructs with up to fifteen fragments or for incorporating fragments from 100 bp up to 10 kb, typically the 2-step is used, where the 2-step reaction requires two separate additions of master mix; one for the exonuclease and annealing step and a second for the polymerase and ligation steps.

The Cell Transformation Module

FIGS. 5A and 5B depict the structure and components of an embodiment of an exemplary reagent cartridge useful in the automated multi-module instrument described therein. In FIG. 5A, reagent cartridge 500 comprises a body 502, which has reservoirs 504. One reservoir 504 is shown empty, and two of the reservoirs have individual tubes (not shown) inserted therein, with individual tube covers 505. Additionally shown are rows of reservoirs into which have been inserted co-joined rows of large tubes 503a, and co-joined rows of small tubes 503b. The co-joined rows of tubes are presented in a strip, with outer flanges 507 that mate on the backside of the outer flange (not shown) with an indentation 509 in the body 502, so as to secure the co-joined rows of tubes (503a and 503b) to the reagent cartridge 500. Shown also is a base 511 of reagent cartridge body 502. Note that the reservoirs 504 in body 502 are shaped generally like the tubes in the co-joined tubes that are inserted into these reservoirs 504.

FIG. 5B depicts the reagent cartridge 500 in FIG. 5A with a row of co-joined large tubes 503a, a row of co-joined small tubes 503b, and one large tube 560 with a cover 505 removed from (i.e., depicted above) the reservoirs 504 of the reagent cartridge 500. Again, the co-joined rows of tubes are presented in a strip, with individual large tubes 561 shown, and individual small tubes 555 shown. Again, each strip of co-joined tubes comprises outer flanges 507 that mate on the backside (not shown) of the outer flange with an indentation 509 in the body 502, to secure the co-joined rows of tubes (503a and 503b) to the reagent cartridge 500. As in FIG. 5A, reagent cartridge body 502 comprises a base 511. Reagent cartridge 500 may be made from any suitable material, including stainless steel, aluminum, or plastics including polyvinyl chloride, cyclic olefin copolymer (COC), polyethylene, polyamide, polypropylene, acrylonitrile butadiene, polycarbonate, polyetheretheketone (PEEK), poly(methyl methylacrylate) (PMMA), polysulfone, and polyurethane, and co-polymers of these and other polymers. Again, if reagent cartridge 500 is disposable, it preferably is made of plastic. In addition, in many embodiments the material used to fabricate the cartridge is thermally-conductive, as reagent cartridge 500 may contact a thermal device (not shown) that heats or cools reagents in the reagent reservoirs 504, including reagents in co-joined tubes. In some embodiments, the thermal device is a Peltier device or thermoelectric cooler.

FIGS. 5C and 5D are top perspective and bottom perspective views, respectively, of an exemplary FTEP device 550 that may be part of (e.g., a component in) reagent cartridge 500 in FIGS. 5A and 5B or may be a stand-alone module; that is, not a part of a reagent cartridge or other module. FIG. 5C depicts an FTEP device 550. The FTEP device 550 has wells that define cell sample inlets 552 and cell sample outlets 554. FIG. 5D is a bottom perspective view of the FTEP device 550 of FIG. 5C. An inlet well 552 and an outlet well 554 can be seen in this view. Also seen in FIG. 5D are the bottom of an inlet 562 corresponding to well 552, the bottom of an outlet 564 corresponding to the outlet well 554, the bottom of a defined flow channel 566 and the bottom of two electrodes 568 on either side of flow channel 566. The FTEP devices may comprise push-pull pneumatic means to allow multi-pass electroporation procedures; that is, cells to electroporated may be “pulled” from the inlet toward the outlet for one pass of electroporation, then be “pushed” from the outlet end of the FTEP device toward the inlet end to pass between the electrodes again for another pass of electroporation. Further, this process may be repeated one to many times. For additional information regarding FTEP devices, see, e.g., U.S. Pat. Nos. 10,435,713; 10,443,074; 10,323,258; and 10,508,288. Further, other embodiments of the reagent cartridge may provide or accommodate electroporation devices that are not configured as FTEP devices, such as those described in U.S. Ser. No. 16/109,156, filed 22 Aug. 2018. For reagent cartridges useful in the present automated multi-module cell processing instruments, see, e.g., U.S. Pat. No. 10,376,889; 10,406,525; 10,576,474; and U.S. Ser. No. 16/749,757, filed 22 Jan. 2020; and Ser. No. 16/827,222, filed 23 Mar. 2020.

Additional details of the FTEP devices are illustrated in FIGS. 5E-5G. Note that in the FTEP devices in FIGS. 5E-5G the electrodes are placed such that a first electrode is placed between an inlet and a narrowed region of the flow channel, and the second electrode is placed between the narrowed region of the flow channel and an outlet. FIG. 5E shows a top planar view of an FTEP device 550 having an inlet 552 for introducing a fluid containing cells and exogenous material into FTEP device 550 and an outlet 554 for removing the transformed cells from the FTEP following electroporation. The electrodes 568 are introduced through channels (not shown) in the device.

FIG. 5F shows a cutaway view from the top of the FTEP device 550, with the inlet 552, outlet 554, and electrodes 568 positioned with respect to a flow channel 566. FIG. 5G shows a side cutaway view of FTEP device 550 with the inlet 552 and inlet channel 572, and outlet 554 and outlet channel 574. The electrodes 568 are positioned in electrode channels 576 so that they are in fluid communication with the flow channel 566, but not directly in the path of the cells traveling through the flow channel 566. Note that the first electrode is placed between the inlet and the narrowed region of the flow channel, and the second electrode is placed between the narrowed region of the flow channel and the outlet. The electrodes 568 in this aspect of the device are positioned in the electrode channels 576 which are generally perpendicular to the flow channel 566 such that the fluid containing the cells and exogenous material flows from the inlet channel 572 through the flow channel 566 to the outlet channel 574, and in the process fluid flows into the electrode channels 576 to be in contact with the electrodes 568. In this aspect, the inlet channel, outlet channel and electrode channels all originate from the same planar side of the device. In certain aspects, however, the electrodes may be introduced from a different planar side of the FTEP device than the inlet and outlet channels.

In the FTEP devices of the disclosure, the toxicity level of the transformation results in greater than 30% viable cells after electroporation, preferably greater than 35%, 40%, 45%, 50%, 55%, 60%, 70%, 75%, 80%, 85%, 90%, 95% or even 99% viable cells following transformation, depending on the cell type and the nucleic acids being introduced into the cells.

The housing of the FTEP device can be made from many materials depending on whether the FTEP device is to be reused, autoclaved, or is disposable, including stainless steel, silicon, glass, resin, polyvinyl chloride, polyethylene, polyamide, polystyrene, polyethylene, polypropylene, acrylonitrile butadiene, polycarbonate, polyetheretheketone (PEEK), polysulfone and polyurethane, co-polymers of these and other polymers. Similarly, the walls of the channels in the device can be made of any suitable material including silicone, resin, glass, glass fiber, polyvinyl chloride, polyethylene, polyamide, polyethylene, polypropylene, acrylonitrile butadiene, polycarbonate, polyetheretheketone (PEEK), polysulfone and polyurethane, co-polymers of these and other polymers. Preferred materials include crystal styrene, cyclo-olefin polymer (COP) and cyclic olephin co-polymers (COC), which allow the device to be formed entirely by injection molding in one piece with the exception of the electrodes and, e.g., a bottom sealing film if present.

The FTEP devices described herein (or portions of the FTEP devices) can be created or fabricated via various techniques, e.g., as entire devices or by creation of structural layers that are fused or otherwise coupled. For example, for metal FTEP devices, fabrication may include precision mechanical machining or laser machining; for silicon FTEP devices, fabrication may include dry or wet etching; for glass FTEP devices, fabrication may include dry or wet etching, powderblasting, sandblasting, or photostructuring; and for plastic FTEP devices fabrication may include thermoforming, injection molding, hot embossing, or laser machining.

The components of the FTEP devices may be manufactured separately and then assembled, or certain components of the FTEP devices (or even the entire FTEP device except for the electrodes) may be manufactured (e.g., using 3D printing) or molded (e.g., using injection molding) as a single entity, with other components added after molding. For example, housing and channels may be manufactured or molded as a single entity, with the electrodes later added to form the FTEP unit. Alternatively, the FTEP device may also be formed in two or more parallel layers, e.g., a layer with the horizontal channel and filter, a layer with the vertical channels, and a layer with the inlet and outlet ports, which are manufactured and/or molded individually and assembled following manufacture.

In specific aspects, the FTEP device can be manufactured using a circuit board as a base, with the electrodes, filter and/or the flow channel formed in the desired configuration on the circuit board, and the remaining housing of the device containing, e.g., the one or more inlet and outlet channels and/or the flow channel formed as a separate layer that is then sealed onto the circuit board. The sealing of the top of the housing onto the circuit board provides the desired configuration of the different elements of the FTEP devices of the disclosure. Also, two to many FTEP devices may be manufactured on a single substrate, then separated from one another thereafter or used in parallel. In certain embodiments, the FTEP devices are reusable and, in some embodiments, the FTEP devices are disposable. In additional embodiments, the FTEP devices may be autoclavable.

The electrodes 508 can be formed from any suitable metal, such as copper, stainless steel, titanium, aluminum, brass, silver, rhodium, gold or platinum, or graphite. One preferred electrode material is alloy 303 (UNS330300) austenitic stainless steel. An applied electric field can destroy electrodes made from of metals like aluminum. If a multiple-use (i.e., non-disposable) flow-through FTEP device is desired—as opposed to a disposable, one-use flow-through FTEP device—the electrode plates can be coated with metals resistant to electrochemical corrosion. Conductive coatings like noble metals, e.g., gold, can be used to protect the electrode plates.

As mentioned, the FTEP devices may comprise push-pull pneumatic means to allow multi-pass electroporation procedures; that is, cells to be electroporated may be “pulled” from the inlet toward the outlet for one pass of electroporation, then be “pushed” from the outlet end of the flow-through FTEP device toward the inlet end to pass between the electrodes again for another pass of electroporation. This process may be repeated one to many times.

Depending on the type of cells to be electroporated (e.g., bacterial, yeast, mammalian) and the configuration of the electrodes, the distance between the electrodes in the flow channel can vary widely. For example, where the flow channel decreases in width, the flow channel may narrow to between 10 μm and 5 mm, or between 25 μm and 3 mm, or between 50 μm and 2 mm, or between 75 μm and 1 mm. The distance between the electrodes in the flow channel may be between 1 mm and 10 mm, or between 2 mm and 8 mm, or between 3 mm and 7 mm, or between 4 mm and 6 mm. The overall size of the FTEP device may be from 3 cm to 15 cm in length, or 4 cm to 12 cm in length, or 4.5 cm to 10 cm in length. The overall width of the FTEP device may be from 0.5 cm to 5 cm, or from 0.75 cm to 3 cm, or from 1 cm to 2.5 cm, or from 1 cm to 1.5 cm.

The region of the flow channel that is narrowed is wide enough so that at least two cells can fit in the narrowed portion side-by-side. For example, a typical bacterial cell is 1 μm in diameter; thus, the narrowed portion of the flow channel of the FTEP device used to transform such bacterial cells will be at least 2 μm wide. In another example, if a mammalian cell is approximately 50 μm in diameter, the narrowed portion of the flow channel of the FTEP device used to transform such mammalian cells will be at least 100 μm wide. That is, the narrowed portion of the FTEP device will not physically contort or “squeeze” the cells being transformed.

In embodiments of the FTEP device where reservoirs are used to introduce cells and exogenous material into the FTEP device, the reservoirs range in volume from 100 μL to 10 mL, or from 500 μL to 75 mL, or from 1 mL to 5 mL. The flow rate in the FTEP ranges from 0.1 mL to 5 mL per minute, or from 0.5 mL to 3 mL per minute, or from 1.0 mL to 2.5 mL per minute. The pressure in the FTEP device ranges from 1-30 psi, or from 2-10 psi, or from 3-5 psi.

To avoid different field intensities between the electrodes, the electrodes should be arranged in parallel. Furthermore, the surface of the electrodes should be as smooth as possible without pin holes or peaks. Electrodes having a roughness Rz of 1 to 10 μm are preferred. In another embodiment of the invention, the flow-through electroporation device comprises at least one additional electrode which applies a ground potential to the FTEP device. Flow-through electroporation devices (either as a stand-alone instrument or as a module in an automated multi-module system) are described in, e.g., U.S. Pat. Nos. 10,435,713; 10,443,074; 10,323,258; and 10,508,288.

Cell Singulation and Enrichment Device

FIG. 6A depicts a solid wall device 6050 and a workflow for singulating cells in microwells in the solid wall device. At the top left of the figure (i), there is depicted solid wall device 6050 with microwells 6052. A section 6054 of substrate 6050 is shown at (ii), also depicting microwells 6052. At (iii), a side cross-section of solid wall device 6050 is shown, and microwells 6052 have been loaded, where, in this embodiment, Poisson or substantial Poisson loading has taken place; that is, each microwell has few, one or no cells. At (iv), workflow 6040 is illustrated where substrate 6050 having microwells 6052 shows microwells 6056 with one cell per microwell, microwells 6057 with no cells in the microwells, and one microwell 6060 with two cells in the microwell. In step 6051, the cells in the microwells are allowed to double approximately 2-150 times to form clonal colonies (v), then editing is allowed to occur 6053.

After editing 6053, many cells in the colonies of cells that have been edited die as a result of the double-strand cuts caused by active editing and there is a lag in growth for the edited cells that do survive but must repair and recover following editing (microwells 6058), where cells that do not undergo editing thrive (microwells 6059) (vi). All cells are allowed to continue grow to establish colonies and normalize 6055, where the colonies of edited cells in microwells 6058 catch up in size and/or cell number with the cells in microwells 6059 that do not undergo editing (vii). Once the cell colonies are normalized, either pooling 6060 of all cells in the microwells can take place, in which case the cells are enriched for edited cells by eliminating the bias from non-editing cells and fitness effects from editing; alternatively, colony growth in the microwells is monitored after editing, and slow growing colonies (e.g., the cells in microwells 6058) are identified and selected 6061 (e.g., “cherry picked”) resulting in even greater enrichment of edited cells.

In growing the cells, the medium used will depend, of course, on the type of cells being edited—e.g., bacterial, yeast or mammalian. For example, medium for yeast cell growth includes LB, SOC, TPD, YPG, YPAD, MEM and DMEM.

A module useful for performing the methods depicted in FIG. 6A is a solid wall isolation, incubation, and normalization (SWIIN) module. FIG. 6B depicts an embodiment of a SWIIN module 650 from an exploded top perspective view. In SWIIN module 650 the retentate member is formed on the bottom of a top of a SWIIN module component and the permeate member is formed on the top of the bottom of a SWIIN module component.

The SWIIN module 650 in FIG. 6B comprises from the top down, a reservoir gasket or cover 658, a retentate member 604 (where a retentate flow channel cannot be seen in this FIG. 6B), a perforated member 601 swaged with a filter (filter not seen in FIG. 6B), a permeate member 608 comprising integrated reservoirs (permeate reservoirs 652 and retentate reservoirs 654), and two reservoir seals 662, which seal the bottom of permeate reservoirs 652 and retentate reservoirs 654. A permeate channel 660a can be seen disposed on the top of permeate member 608, defined by a raised portion 676 of serpentine channel 660a, and ultrasonic tabs 664 can be seen disposed on the top of permeate member 608 as well. The perforations that form the wells on perforated member 601 are not seen in this FIG. 6B; however, through-holes 666 to accommodate the ultrasonic tabs 664 are seen. In addition, supports 670 are disposed at either end of SWIIN module 650 to support SWIIN module 650 and to elevate permeate member 608 and retentate member 604 above reservoirs 652 and 654 to minimize bubbles or air entering the fluid path from the permeate reservoir to serpentine channel 660a or the fluid path from the retentate reservoir to serpentine channel 660b (neither fluid path is seen in this FIG. 6B). Also seen is a gasket 658, which covers the permeate and retentate reservoir access apertures 632a, 632b, 632c, and 632d, as well as pneumatic ports 633a, 633b, 633c and 633d.

In this FIG. 6B, it can be seen that the serpentine channel 660a that is disposed on the top of permeate member 608 traverses permeate member 608 for most of the length of permeate member 608 except for the portion of permeate member 608 that comprises permeate reservoirs 652 and retentate reservoirs 654 and for most of the width of permeate member 608. As used herein with respect to the distribution channels in the retentate member or permeate member, “most of the length” means about 95% of the length of the retentate member or permeate member, or about 90%, 85%, 80%, 75%, or 70% of the length of the retentate member or permeate member. As used herein with respect to the distribution channels in the retentate member or permeate member, “most of the width” means about 95% of the width of the retentate member or permeate member, or about 90%, 85%, 80%, 75%, or 70% of the width of the retentate member or permeate member.

In this embodiment of a SWIIN module, the perforated member includes through-holes to accommodate ultrasonic tabs disposed on the permeate member. Thus, in this embodiment the perforated member is fabricated from 316 stainless steel, and the perforations form the walls of microwells while a filter or membrane is used to form the bottom of the microwells. Typically, the perforations (microwells) are approximately 150 μm-200 μm in diameter, and the perforated member is approximately 125 μm deep, resulting in microwells having a volume of approximately 2.5 nl, with a total of approximately 200,000 microwells. The distance between the microwells is approximately 279 μm center-to-center. Though here the microwells have a volume of approximately 2.5 nl, the volume of the microwells may be from 1 to 25 nl, or preferably from 2 to 10 nl, and even more preferably from 2 to 4 nl. As for the filter or membrane, like the filter described previously, filters appropriate for use are solvent resistant, contamination free during filtration, and are able to retain the types and sizes of cells of interest. For example, in order to retain small cell types such as bacterial cells, pore sizes can be as low as 0.10 μm, however for other cell types (e.g., such as for mammalian cells), the pore sizes can be as high as 10.0 μm-20.0 μm or more. Indeed, the pore sizes useful in the cell concentration device/module include filters with sizes from 0.10 μm, 0.11 μm, 0.12 μm, 0.13 μm, 0.14 μm, 0.15 μm, 0.16 μm, 0.17 μm, 0.18 μm, 0.19 μm, 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable material including cellulose mixed ester (cellulose nitrate and acetate) (CME), polycarbonate (PC), polyvinylidene fluoride (PVDF), polyethersulfone (PES), polytetrafluoroethylene (PTFE), nylon, or glass fiber.

The cross-section configuration of the mated serpentine channel may be round, elliptical, oval, square, rectangular, trapezoidal, or irregular. If square, rectangular, or another shape with generally straight sides, the cross section may be from about 2 mm to 15 mm wide, or from 3 mm to 12 mm wide, or from 5 mm to 10 mm wide. If the cross section of the mated serpentine channel is generally round, oval or elliptical, the radius of the channel may be from about 3 mm to 20 mm in hydraulic radius, or from 5 mm to 15 mm in hydraulic radius, or from 8 mm to 12 mm in hydraulic radius.

Serpentine channels 660a and 660b can have approximately the same volume or a different volume. For example, each “side” or portion 660a, 660b of the serpentine channel may have a volume of, e.g., 2 mL, or serpentine channel 660a of permeate member 608 may have a volume of 2 mL, and the serpentine channel 660b of retentate member 604 may have a volume of, e.g., 3 mL. The volume of fluid in the serpentine channel may range from about 2 mL to about 80 mL, or about 4 mL to 60 mL, or from 5 mL to 40 mL, or from 6 mL to 20 mL (note these volumes apply to a SWIIN module comprising a, e.g., 50-500K perforation member). The volume of the reservoirs may range from 5 mL to 50 mL, or from 7 mL to 40 mL, or from 8 mL to 30 mL or from 10 mL to 20 mL, and the volumes of all reservoirs may be the same or the volumes of the reservoirs may differ (e.g., the volume of the permeate reservoirs is greater than that of the retentate reservoirs).

The serpentine channel portions 660a and 660b of the permeate member 608 and retentate member 604, respectively, are approximately 200 mm long, 130 mm wide, and 4 mm thick, though in other embodiments, the retentate and permeate members can be from 75 mm to 400 mm in length, or from 100 mm to 300 mm in length, or from 150 mm to 250 mm in length; from 50 mm to 250 mm in width, or from 75 mm to 200 mm in width, or from 100 mm to 150 mm in width; and from 2 mm to 15 mm in thickness, or from 4 mm to 10 mm in thickness, or from 5 mm to 8 mm in thickness. Embodiments the retentate (and permeate) members may be fabricated from PMMA (poly(methyl methacrylate) or other materials may be used, including polycarbonate, cyclic olefin co-polymer (COC), glass, polyvinyl chloride, polyethylene, polyamide, polypropylene, polysulfone, polyurethane, and co-polymers of these and other polymers. Preferably at least the retentate member is fabricated from a transparent material so that the cells can be visualized (see, e.g., FIG. 6E and the description thereof). For example, a video camera may be used to monitor cell growth by, e.g., density change measurements based on an image of an empty well, with phase contrast, or if, e.g., a chromogenic marker, such as a chromogenic protein, is used to add a distinguishable color to the cells. Chromogenic markers such as blitzen blue, dreidel teal, virginia violet, vixen purple, prancer purple, tinsel purple, maccabee purple, donner magenta, cupid pink, seraphina pink, scrooge orange, and leor orange (the Chromogenic Protein Paintbox, all available from ATUM (Newark, Calif.)) obviate the need to use fluorescence, although fluorescent cell markers, fluorescent proteins, and chemiluminescent cell markers may also be used.

Because the retentate member preferably is transparent, colony growth in the SWIIN module can be monitored by automated devices such as those sold by JoVE (ScanLag™ system, Cambridge, Mass.) (also see Levin-Reisman, et al., Nature Methods, 7:737-39 (2010)). Automated colony pickers may be employed, such as those sold by, e.g., TECAN (Pickolo™ system, Mannedorf, Switzerland); Hudson Inc. (RapidPick™, Springfield, N.J.); Molecular Devices (QPix 400™ system, San Jose, Calif.); and Singer Instruments (PIXL™ system, Somerset, UK).

Due to the heating and cooling of the SWIIN module, condensation may accumulate on the retentate member which may interfere with accurate visualization of the growing cell colonies. Condensation of the SWIIN module 650 may be controlled by, e.g., moving heated air over the top of (e.g., retentate member) of the SWIIN module 650, or by applying a transparent heated lid over at least the serpentine channel portion 660b of the retentate member 604. See, e.g., FIG. 6E and the description thereof infra.

In SWIIN module 650 cells and medium—at a dilution appropriate for Poisson or substantial Poisson distribution of the cells in the microwells of the perforated member—are flowed into serpentine channel 660b from ports in retentate member 604, and the cells settle in the microwells while the medium passes through the filter into serpentine channel 660a in permeate member 608. The cells are retained in the microwells of perforated member 601 as the cells cannot travel through filter 603. Appropriate medium may be introduced into permeate member 608 through permeate ports 611. The medium flows upward through filter 603 to nourish the cells in the microwells (perforations) of perforated member 601. Additionally, buffer exchange can be effected by cycling medium through the retentate and permeate members. In operation, the cells are deposited into the microwells, are grown for an initial, e.g., 2-100 doublings, editing may be induced by, e.g., raising the temperature of the SWIIN to 42° C. to induce a temperature-inducible promoter or by removing growth medium from the permeate member and replacing the growth medium with a medium comprising a chemical component that induces an inducible promoter.

Once editing has taken place, the temperature of the SWIIN may be decreased, or the inducing medium may be removed and replaced with fresh medium lacking the chemical component thereby de-activating the inducible promoter. The cells then continue to grow in the SWIIN module 650 until the growth of the cell colonies in the microwells is normalized. For the normalization protocol, once the colonies are normalized, the colonies are flushed from the microwells by applying fluid or air pressure (or both) to the permeate member serpentine channel 660a and thus to filter 603 and pooled. Alternatively, if cherry picking is desired, the growth of the cell colonies in the microwells is monitored, and slow-growing colonies are directly selected; or, fast-growing colonies are eliminated.

FIG. 6C is a top perspective view of a SWIIN module with the retentate and perforated members in partial cross section. In this FIG. 6C, it can be seen that serpentine channel 660a is disposed on the top of permeate member 608 is defined by raised portions 676 and traverses permeate member 608 for most of the length and width of permeate member 608 except for the portion of permeate member 608 that comprises the permeate and retentate reservoirs (note only one retentate reservoir 652 can be seen). Moving from left to right, reservoir gasket 658 is disposed upon the integrated reservoir cover 678 (cover not seen in this FIG. 6C) of retentate member 604. Gasket 658 comprises reservoir access apertures 632a, 632b, 632c, and 632d, as well as pneumatic ports 633a, 633b, 633c and 633d. Also at the far left end is support 670. Disposed under permeate reservoir 652 can be seen one of two reservoir seals 662. In addition to the retentate member being in cross section, the perforated member 601 and filter 603 (filter 603 is not seen in this FIG. 6C) are in cross section. Note that there are a number of ultrasonic tabs 664 disposed at the right end of SWIIN module 650 and on raised portion 676 which defines the channel turns of serpentine channel 660a, including ultrasonic tabs 664 extending through through-holes 666 (not seen in this FIG. 6C but see FIG. 6B) of perforated member 601. There is also a support 670 at the end distal reservoirs 652, 654 of permeate member 608.

FIG. 6D is a side perspective view of an assembled SWIIIN module 650, including, from right to left, reservoir gasket 658 disposed upon integrated reservoir cover 678 (not seen) of retentate member 604. Gasket 658 may be fabricated from rubber, silicone, nitrile rubber, polytetrafluoroethylene, a plastic polymer such as polychlorotrifluoroethylene, or other flexible, compressible material. Gasket 658 comprises reservoir access apertures 632a, 632b, 632c, and 632d, as well as pneumatic ports 633a, 633b, 633c and 633d. Also at the far-left end is support 670 of permeate member 608. In addition, permeate reservoir 652 can be seen, as well as one reservoir seal 662. At the far-right end is a second support 670.

Imaging of cell colonies growing in the wells of the SWIIN is desired in most implementations for, e.g., monitoring both cell growth and device performance and imaging is necessary for cherry-picking implementations. Real-time monitoring of cell growth in the SWIIN requires backlighting, retentate plate (top plate) condensation management and a system-level approach to temperature control, air flow, and thermal management. In some implementations, imaging employs a camera or CCD device with sufficient resolution to be able to image individual wells. For example, in some configurations a camera with a 9-pixel pitch is used (that is, there are 9 pixels center-to-center for each well). Processing the images may, in some implementations, utilize reading the images in grayscale, rating each pixel from low to high, where wells with no cells will be brightest (due to full or nearly-full light transmission from the backlight) and wells with cells will be dim (due to cells blocking light transmission from the backlight). After processing the images, thresholding is performed to determine which pixels will be called “bright” or “dim”, spot finding is performed to find bright pixels and arrange them into blocks, and then the spots are arranged on a hexagonal grid of pixels that correspond to the spots. Once arranged, the measure of intensity of each well is extracted, by, e.g., looking at one or more pixels in the middle of the spot, looking at several to many pixels at random or pre-set positions, or averaging X number of pixels in the spot. In addition, background intensity may be subtracted. Thresholding is again used to call each well positive (e.g., containing cells) or negative (e.g., no cells in the well). The imaging information may be used in several ways, including taking images at time points for monitoring cell growth. Monitoring cell growth can be used to, e.g., remove the “muffin tops” of fast-growing cells followed by removal of all cells or removal of cells in “rounds” as described above, or recover cells from specific wells (e.g., slow-growing cell colonies); alternatively, wells containing fast-growing cells can be identified and areas of UV light covering the fast-growing cell colonies can be projected (or rastered with shutters) onto the SWIIN to irradiate or inhibit growth of those cells. Imaging may also be used to assure proper fluid flow in the serpentine channel 660.

FIG. 6E depicts the embodiment of the SWIIN module in FIGS. 6B-6D further comprising a heat management system including a heater and a heated cover. The heated cover facilitates the condensation management that is required for imaging. Assembly 698 comprises a SWIIN module 650 seen lengthwise in cross section, where one permeate reservoir 652 is seen. Disposed immediately upon SWIIN module 650 is cover 694 and disposed immediately below SWIIN module 650 is backlight 680, which allows for imaging. Beneath and adjacent to the backlight and SWIIN module is insulation 682, which is disposed over a heatsink 684. In this FIG. 6E, the fins of the heatsink would be in-out of the page. In addition there is also axial fan 686 and heat sink 688, as well as two thermoelectric coolers 692, and a controller 690 to control the pneumatics, thermoelectric coolers, fan, solenoid valves, etc. The arrows denote cool air coming into the unit and hot air being removed from the unit. It should be noted that control of heating allows for growth of many different types of cells as well as strains of cells that are, e.g., temperature sensitive, etc., and allows use of temperature-sensitive promoters. Temperature control allows for protocols to be adjusted to account for differences in transformation efficiency, cell growth and viability. For more details regarding solid wall isolation incubation and normalization devices see USPNs 10,533,152; 10,550,363; 10,532,324; 10,625,212; 10,633,626; and 10,633,627; and U.S. Ser. No. 16/693,630, filed 25 Nov. 2019; Ser. No. 16/823,269, filed 18 Mar. 2020; Ser. No. 16/820,292, filed 16 Mar. 2020; Ser. No. 16/820,324, filed 16 Mar. 2020; and Ser. No. 16/686,066, filed 15 Nov. 2019.

Use of the Automated Multi-Module Yeast Cell Processing Instrument

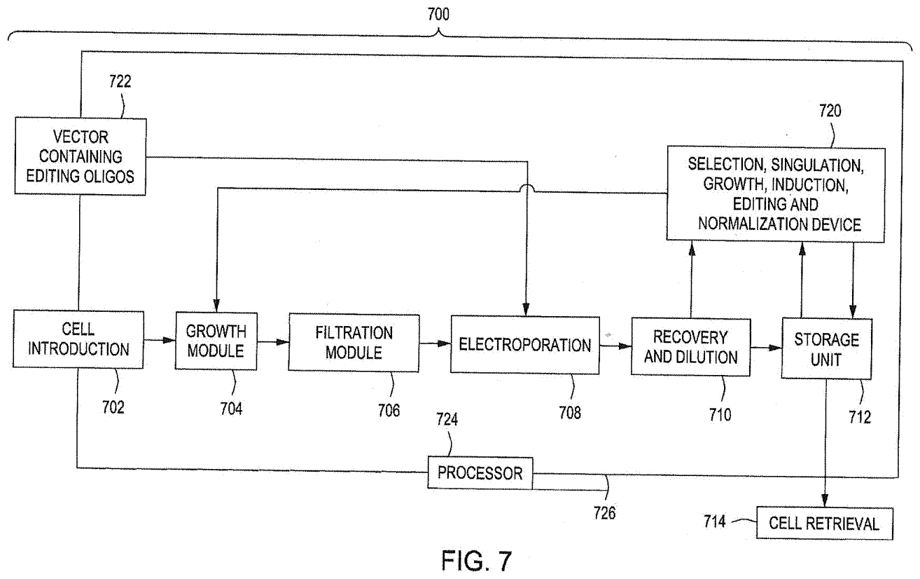

FIG. 7 illustrates an embodiment of a multi-module cell processing instrument. This embodiment depicts an exemplary system that performs recursive gene editing on a cell population. The cell processing instrument 700 may include a housing 726, a reservoir for storing cells to be transformed or transfected 702, and a cell growth module (comprising, e.g., a rotating growth vial) 704. The cells to be transformed are transferred from a reservoir to the cell growth module to be cultured until the cells hit a target OD. Once the cells hit the target OD, the growth module may cool or freeze the cells for later processing or transfer the cells to a cell concentration module 706 where the cells are subjected to buffer exchange and rendered electrocompetent, and the volume of the cells may be reduced substantially. Once the cells have been concentrated to an appropriate volume, the cells are transferred to electroporation device 708. In addition to the reservoir for storing cells 702, the multi-module cell processing instrument includes a reservoir for storing the vector pre-assembled with editing oligonucleotide cassettes 722. The pre-assembled nucleic acid vectors are transferred to the electroporation device 708, which already contains the cell culture grown to a target OD. In the electroporation device 708, the nucleic acids are electroporated into the cells. Following electroporation, the cells are transferred into an optional recovery module 710, where the cells recover briefly post-transformation.

After recovery, the cells may be transferred to a storage module 712, where the cells can be stored at, e.g., 4° C. for later processing 714, or the cells may be diluted and transferred to a selection/singulation/growth/induction/editing/normalization (SWIIN) module 720. In the SWIIN 720, the cells are arrayed such that there is an average of one cell per microwell. The arrayed cells may be in selection medium to select for cells that have been transformed or transfected with the editing vector(s). Once singulated, the cells grow through 2-50 doublings and establish colonies. Once colonies are established, editing is induced by providing conditions (e.g., temperature, addition of an inducing or repressing chemical) to induce editing. Once editing is initiated and allowed to proceed, the cells are allowed to grow to terminal size (e.g., normalization of the colonies) in the microwells and then the cells are treated to conditions that cure the editing vector from this round. Once cured, the cells can be flushed out of the microwells and pooled, then transferred to the storage (or recovery) unit 712 or can be transferred back to the growth module 704 for another round of editing. In between pooling and transfer to a growth module, there typically is one or more additional steps, such as cell recovery, medium exchange (rendering the cells electrocompetent), cell concentration (typically concurrently with medium exchange by, e.g., filtration. Note that the selection/singulation/growth/induction/editing/normalization and editing modules may be the same module, where all processes are performed in, e.g., a solid wall device, or selection and/or dilution may take place in a separate vessel before the cells are transferred to the solid wall singulation/growth/induction/editing/normalization/editing module (solid wall device). Similarly, the cells may be pooled after normalization, transferred to a separate vessel, and cured in the separate vessel. As an alternative to singulation in, e.g., a solid wall device, the transformed cells may be grown in—and editing can be induced in—bulk liquid as described above in relation to FIGS. 1G and 1H above. Once the putatively-edited cells are pooled, they may be subjected to another round of editing, beginning with growth, cell concentration and treatment to render electrocompetent, and transformation by yet another donor nucleic acid in another editing cassette via the electroporation module 708.

In electroporation device 708, the cells selected from the first round of editing are transformed by a second set of editing oligos (or other type of oligos) and the cycle is repeated until the cells have been transformed and edited by a desired number of, e.g., editing cassettes. The multi-module cell processing instrument exemplified in FIG. 7 is controlled by a processor 724 configured to operate the instrument based on user input or is controlled by one or more scripts including at least one script associated with the reagent cartridge. The processor 724 may control the timing, duration, and temperature of various processes, the dispensing of reagents, and other operations of the various modules of the instrument 700. For example, a script or the processor may control the dispensing of cells, reagents, vectors, and editing oligonucleotides; which editing oligonucleotides are used for cell editing and in what order; the time, temperature and other conditions used in the recovery and expression module, the wavelength at which OD is read in the cell growth module, the target OD to which the cells are grown, and the target time at which the cells will reach the target OD. In addition, the processor may be programmed to notify a user (e.g., via an application) as to the progress of the cells in the automated multi-module cell processing instrument.

It should be apparent to one of ordinary skill in the art given the present disclosure that the process described may be recursive and multiplexed; that is, cells may go through the workflow described in relation to FIG. 7, then the resulting edited culture may go through another (or several or many) rounds of additional editing (e.g., recursive editing) with different editing vectors. For example, the cells from round 1 of editing may be diluted and an aliquot of the edited cells edited by editing vector A may be combined with editing vector B, an aliquot of the edited cells edited by editing vector A may be combined with editing vector C, an aliquot of the edited cells edited by editing vector A may be combined with editing vector D, and so on for a second round of editing. After round two, an aliquot of each of the double-edited cells may be subjected to a third round of editing, where, e.g., aliquots of each of the AB-, AC-, AD-edited cells are combined with additional editing vectors, such as editing vectors X, Y, and Z. That is that double-edited cells AB may be combined with and edited by vectors X, Y, and Z to produce triple-edited edited cells ABX, ABY, and ABZ; double-edited cells AC may be combined with and edited by vectors X, Y, and Z to produce triple-edited cells ACX, ACY, and ACZ; and double-edited cells AD may be combined with and edited by vectors X, Y, and Z to produce triple-edited cells ADX, ADY, and ADZ, and so on. In this process, many permutations and combinations of edits can be executed, leading to very diverse cell populations and cell libraries.

In any recursive process, it is advantageous to “cure” the previous engine and editing vectors (or single engine+editing vector in a single vector system). “Curing” is the process in which one or more vectors used in the prior round of editing is eliminated from the transformed cells as described in detail above in relation to FIGS. 1A-1I. Curing can be accomplished by, e.g., cleaving the vector(s) using a curing plasmid thereby rendering the editing and/or engine vector (or single, combined vector) nonfunctional; diluting the vector(s) in the cell population via cell growth (that is, the more growth cycles the cells go through, the fewer daughter cells will retain the editing or engine vector(s)), or by, e.g., utilizing a heat-sensitive origin of replication on the editing or engine vector (or combined engine+editing vector). The conditions for curing will depend on the mechanism used for curing; that is, in this example, how the curing plasmid cleaves the editing and/or engine vector.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: Schematic for Digital Image Information Storage

A set of DNA encoding a digital bitmap image representative of Mona Lisa is encoded, using methods known in the art, and cloned to a group of plasmids. This population of plasmids is then transformed into an MG1655 strain containing an insertion of AGAGCG 6-nt insertion at position 96 of rpoA, which provides resistance to coumaric acid and sensitivity to isobutyric acid. A second set of DNA encoding a digital bitmap image representation of Starry Night is encoded, using methods known in the art, and cloned to a group of plasmids. This population of plasmids is that transformed into an MG1655 strain containing a single nucleotide polymorphism mutation in rpoC A104G, which provides growth tolerance to isobutyric acid and sensitivity to coumaric acid. The population can then be mixed together and with other wild-type or engineered tolerance populations to generate a mixed pool containing digital information. Upon needing to extract or otherwise enrich for the digital information of interest, the pool is grown in the presence of the selective compound. The population with tolerance will enrich in the media containing the compound while the other populations will deplete away. After 24 hours of growth, the media is sampled, and DNA is extracted, and the sequences of interest are amplified using a general primer set flanking the region of containing the inserts. The amplification is sequenced and the encoded information is reconstructed from the obtained sequences. FIG. 1 is an schematic illustrating such methods for Boolean storage of information in a population of microbes that carry information segmented amongst cell populations that have been raised to have a tolerance to a set of compounds.

### Example II: Schematic for Digital Text Information Storage

Seven collections of designs were generated using the six constitutive promoter sequences with a spacer sequence followed by a high efficiency ribosome binding site (RBS) and a seventh design collection containing a triple stop codon with 2 of these per CDS:

TABLE 1

| Library | | |
|---|---|---|
| Name | Insert Sequence | Designs |
| ON127SPG1 | Ttgacggctagctcagtcctaggtacagtgc | 4,200 |
| | tagctaagtaggtacgtaaggaggtgataa | |
| | (SEQ ID NO: 1) | |
| ON127SPG2 | Ctgatagctagctcagtcctagggattatgc | 4,200 |
| | tagctaagtaggtacgtaaggaggtgataa | |
| | (SEQ ID NO: 2) | |
| ON127SPG3 | Ttgacagctagctcagtcctagggactatgc | 4,200 |
| | tagctaagtaggtacgtaaggaggtgataa | |
| | (SEQ ID NO: 3) | |
| ON127SPG4 | Tttacggctagctcagtcctaggtatagtgc | 4,203 |
| | tagctaagtaggtacgtaaggaggtgataa | |
| | (SEQ ID NO: 4) | |
| ON127SPG5 | Tttacagctagctcagtcctaggtattatgc | 4,202 |
| | tagctaagtaggtacgtaaggaggtgataa | |
| | (SEQ ID NO: 5) | |
| ON127SPG13 | Ttgacagctagctcagtcctaggtataatgc | 4,200 |
| | tagctaagtaggtacgtaaggaggtgataa | |
| | (SEQ ID NO: 6) | |
| ON127SPG6 | taatgataa | 8,248 |

For the six promoter-spacer-RBS sequences, each sequence was inserted directly in front of the start codon for each coding sequence (CDS) in the E. coli genome (strain MG1655; NCBI accession U00096.3), when accessible by the MAD7 nuclease given PAM site placement, resulting in a total of designs shown in table 1 out of a possible 4,240 CDSs per design collection. For the triple stop codon collection, the sequence was inserted into the 12^{th} position of every CDS in the E. coli genome, resulting in a total of designs shown in table 1 out of 8,480 possible designs.

Designs were generated using an automated approach for combining a spacer crRNA, the repair template containing flanking homology arms to the repair site and the insertion sequence between them, either the promoter-spacer-RBS or the triple stop, and a unique barcode sequence that allows tracking the design in the final cell pool. The cassettes were manufactured, assembled, and editing of the library pool was carried out as previously described (CREATE). Edits were verified by amplifying the region containing the barcode and sequencing the barcodes in the population to assess homogeneity, and additionally by calculating the edited fraction of the collection pool by sequence verification of edits from isolate colonies. The edited collection pools were then additionally pooled together to form a library pool containing a constitutive laddered promoter in front of each gene and a knockout of each gene in the MG1655 strain. Approximately 10⁶ cells from each cell pool were then used to inoculate triplicate baffled shaker flasks containing 50 ml of M9 minimal media supplemented with 1% glucose alone; terrific broth (TB); Luria broth (LB); M9+glucose with 1 g/L furfural or 1 g/L vanillin or 1 g/L syringic acid or 1 g/L 5-hydroxymethylfurfural (5-HMF); or TB with 3 g/L furfural or 3 g/L vanillin.

### Example III: Schematic for Boolean Retrieval of Digital Information Storage

The cell pool generated in the previous example was typically grown to saturation under the aforementioned conditions and 500 ul was collected and the cell pellet was processed to purify the total cell DNA.

The barcode was amplified and sequenced using Illumina NextSeq sequencing. The barcodes quantities were compared to the aggregate population of barcodes by calculating the log₁₀ of the design representation of each design

$${log}_{10} ( \frac{\frac{design count}{\sum design counts}}{\frac{1}{total designs}} )$$

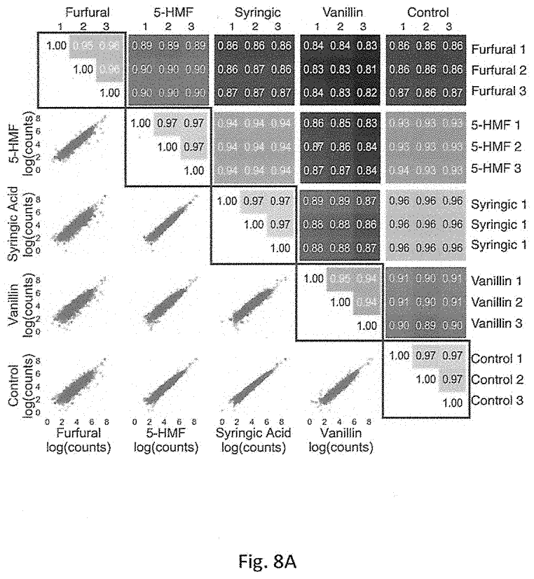

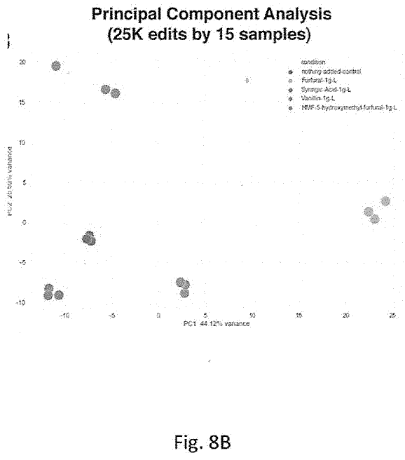

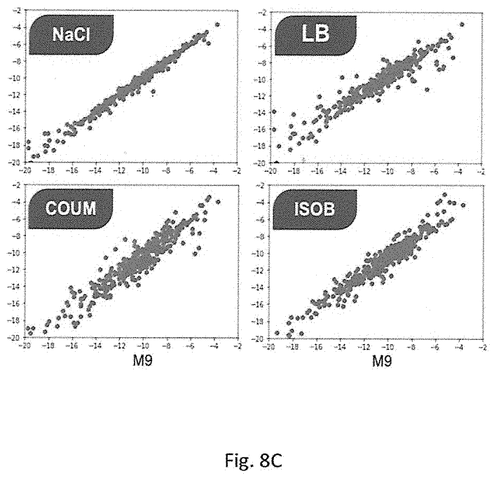

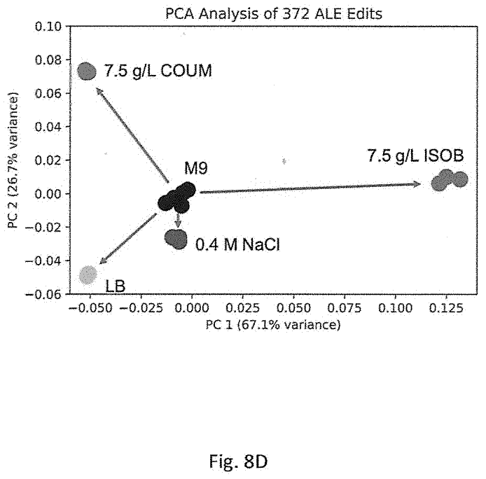

This log₁₀ design representation (1DR) was calculated for each design in the replicates of the media with and without the presence of growth inhibition. Designs that enrich are those for which the 1DR>0 while designs that deplete are those for which the 1DR<0, irrespective of the presence of growth inhibitors. The behavior of each design in each growth inhibitor can be tracked by calculating the difference between the design 1DR in the presence and absence of the growth inhibitor. Edits that enrich in growth inhibitors compared to those same designs in media without inhibitors such that 1DR_{design,inhibitor}>1DR_{design,control} whereas those that deplete are such that 1DR_{design,inhibitor}<1DR_{design,control}. Typically plots are made showing the average of replicate 1DRs of the growth inhibitor of each design on the y axis and average of replicate 1DRs of the control media of each design on the x axis, showing a distribution where each data point represents a single design and those data points that deviate significantly away from the best fit line are representative of the enrichers and depletors, being associated with some promoter-spacer-RBS insert or gene knock out (FIG. 8A). FIG. 8A illustrates the Pearson correlation matrix between tested compounds and replicates and the scatter matrix of each. Each point on the scatter plot is representative of a design in the pool. Points deviating from the best fit line above are enriching designs, and those below are depleting designs.

The overall profile of this distribution was further condensed using principal component analysis (PCA) which leverages Eigenvector decomposition of the reduced covariance matrix to identify trends in the population. Applying PCA to the distribution of 1DR for all designs shows a distinct grouping of replicates separated by the applied inhibitor. This reproducible model of the population response to applied growth pressure shows that if the same population was subjected to a growth pressure of an unknown compound chosen from the set of tested compounds (e.g. chosen here from furfural, vanillin, syringic acid, and hydroxymethylfurfural), the PCA of the 1DR of the population could be used to identify the compound (FIG. 8B). FIG. 8B illustrates the principal component analysis of the bulk response in the presence of different growth inhibitors. Placement of the library response in the decomposition indicates a response identifying that particular compound.

### Example IV: Schematic of Reproducible Microbe Populations for Digital Media Storage

A database of mutations generated with the methods described herein was mined for single nucleotide polymorphisms, short deletions, or short insertions and were converted into engineered designs by leveraging the same automated approach described above. The compounds selected for growth inhibition were coumaric acid and isobutryic acid. The designs from the database were again manufactured and assembled to plasmids and the genome edit procedure was carried out as typical. The library of cells then contained 372 individual edits per cell in a pool of cells. Further, in this library a set of knockouts were also created for each of these genes as a proxy for testing loss of function. This library pool was then grown in M9+glucose, LB, or M9+gluclose with 7.5 g/L p-coumaric acid or 7.5 g/L of isobutryic acid until saturation. 500 ul of the saturated material was then collected and the genomic DNA was purified, with barcode sequencing achieving the same metrics as used above (FIG. 8C). FIG. 8C is a Log10 Design Representation (1DR) of M9 barcode sequence counts on the x-axis and the titled inhibitor or media on the y-axis. Each point on the scatter plot is the average 1DR of triplicate measures in the media and is representative of a single design.

Again, PCA was applied to the 1DR for each population of designs and a “fingerprint” of the response was identified by the placement within the Eigenspace. Thus, if the cell population was grown in the presence of one of the compounds selected in a blind experiment, the compound could be identified by the placement in the Eigenspace or by the spectrum of design responses (FIG. 8D). FIG. 8D is a principal component analysis (PCA) of the bulk population responses to the presence of the inhibitor compounds.

Further, a subset of designs within the experimental test shows significant enrichment or depletion in the presence of the growth inhibitor (e.g., designs that are two standard deviations away from the best fit line in the presence of furfural). These designs are then associated to edited variants in the population that allow for compound tolerance or sensitivity. These mutations can then be recapitulated in the MG1655 genome to generate a population of E. coli that are specifically sensitive or tolerant to a growth inhibitor, equating this inhibitor then to generating a synthetic version of an antibiotic, wherein a bacteria is made sensitive to a compound and whereby mutations can be made or introduced on a plasmid to also make that bacteria tolerant or a second set of bacteria tolerant to that compound. Thus a pool of bacteria could be selected by making them sensitive to compounds typically found in the environment or compounds that are biodegradable or compounds that are otherwise bio-orthogonal when ingested such that bacteria could be made sensitive to eating a certain harmless compound. Thus, a colony of bacteria encoding bioproduction of small molecule or therapeutic compounds could be introduced in the environment or in the microbiome of the human gut for example that could be eliminated or engineered by the presence of an otherwise natural or harmless compound.

### Example V: Boolean Retrieval of Information from a Population of Microbes that Carry Information Segmented Across Populations that have been Raised to have Tolerance to a Set of Compounds

Digital information storage on the sequence of DNA is of interest due to its extremely high information density and long life of storage under even suboptimal conditions. DNA has been maintained as the principal source of heritable information for billions of years and living systems have evolved many methods of exquisite replication fidelity and speed. Many alternative methods have been developed for optimal encoding of digital information in sequences of DNA including base₄ representations, fountain coding, and coding within polynucleotide transitions. Leveraging the engineered populations that are sensitive or tolerant to select compounds and the ability to insert DNA sequences in high throughput, DNA sequences encoding three distinct QR codes linking to three paintings were generated using a DNA fountain approach with 45 bp DNA inserts into safe sites without biological impact on the bacterial growth (Table 2).

TABLE 2

| Title | QR code |
|---|---|
| Mona Lisa | [symbol] |
| Whistler’s Mother | [symbol] |
| The Starry Night | [symbol] |

The DNA was encoded with redundancy to account for errors after first using run-length encoding of the bits encoding the QR code (0=white, 1=black). The DNA were designed as inserts using the same architecture as described above for identifying biological tolerance mutations. These designs will then be manufactured and transformed to make these insertions in the genomes of pools of bacteria. The bacterial pools will be distinct for each of the 3 text files. The first pool encoding a QR code linking to the Mona Lisa will be introduced into the cell population which has sensitivity to furfural and tolerance to p-coumaric acid and vanillin.

The second pool encoding a QR code linking to Whistler's Mother will be introduced into the cell population which has sensitivity to vanillin and tolerance to coumaric acid and furfural. The third pool encoding a QR code linking to The Starry Night will be introduced into the cell population which has sensitivity to p-coumaric acid and tolerance to furfural and vanillin. These pools will then be mixed into a single culture. Selection of each text file will then be done by inoculating a new culture in the presence of the growth inhibitor and a growth inhibition concentration (1 g/L) such that the selection will occur only for the population of interest and sequencing the pool at the insert sites and reassembling the enriched sequence to recombine the original QR code. OR operations on the molecular database will be carried out by addition of only the compound which gives the result of interest.

Mona Lisa will be selected by growth in the presence of coumaric acid and vanillin. Mona Lisa OR Whistler's Mother will be selected by presence of coumaric acid. Here the painting's name is used as a metadata association between the QR code link and the selection compound, however, this selection profile could extend to associating compounds to any metadata such as year produced, or other descriptors and the same selection scheme could be applied to the pool. Thus, a pool of cells encoding a database of digital information could be housed within a natural reservoir or environment that information could be specifically extracted by inoculation and growth in a set of compounds.

### Example VI: Fully-Automated Singleplex RGN-directed Editing Run

Singleplex automated genomic editing using MAD7 nuclease was successfully performed with an automated multi-module instrument of the disclosure. See U.S. Pat. No. 9,982,279; and U.S. Ser. No. 16/024,831 filed 30 Jun. 2018; Ser. No. 16/024,816 filed 30 Jun. 2018; Ser. No. 16/147,353 filed 28 Sep. 2018; Ser. No. 16/147,865 filed 30 Sep. 2018; and Ser. No. 16/147,871 filed 30 Jun. 2018.

An ampR plasmid backbone and a lacZ_F172* editing cassette were assembled via Gibson Assembly® into an “editing vector” in an isothermal nucleic acid assembly module included in the automated instrument. lacZ_F172 functionally knocks out the lacZ gene. “lacZ_F172*” indicates that the edit happens at the 172nd residue in the lacZ amino acid sequence. Following assembly, the product was de-salted in the isothermal nucleic acid assembly module using AMPure beads, washed with 80% ethanol, and eluted in buffer. The assembled editing vector and recombineering-ready, electrocompetent E. Coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module), and allowed to recover in SOC medium containing chloramphenicol. Carbenicillin was added to the medium after 1 hour, and the cells were allowed to recover for another 2 hours. After recovery, the cells were held at 4° C. until recovered by the user.

After the automated process and recovery, an aliquot of cells was plated on MacConkey agar base supplemented with lactose (as the sugar substrate), chloramphenicol and carbenicillin and grown until colonies appeared. White colonies represented functionally edited cells, purple colonies represented un-edited cells. All liquid transfers were performed by the automated liquid handling device of the automated multi-module cell processing instrument.

The result of the automated processing was that approximately 1.0E⁻⁰³ total cells were transformed (comparable to conventional benchtop results), and the editing efficiency was 83.5%. The lacZ_172 edit in the white colonies was confirmed by sequencing of the edited region of the genome of the cells. Further, steps of the automated cell processing were observed remotely by webcam and text messages were sent to update the status of the automated processing procedure.

### Example VII: Fully-Automated Recursive Editing Run

Recursive editing was successfully achieved using the automated multi-module cell processing system. An ampR plasmid backbone and a lacZ_V10* editing cassette were assembled via Gibson Assembly® into an “editing vector” in an isothermal nucleic acid assembly module included in the automated system. Similar to the lacZ_F172 edit, the lacZ_V10 edit functionally knocks out the lacZ gene. “lacZ_V10” indicates that the edit happens at amino acid position 10 in the lacZ amino acid sequence. Following assembly, the product was de-salted in the isothermal nucleic acid assembly module using AMPure beads, washed with 80% ethanol, and eluted in buffer. The first assembled editing vector and the recombineering-ready electrocompetent E. Coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module) allowed to recover in SOC medium containing chloramphenicol. Carbenicillin was added to the medium after 1 hour, and the cells were grown for another 2 hours. The cells were then transferred to a centrifuge module and a media exchange was then performed. Cells were resuspended in TB containing chloramphenicol and carbenicillin where the cells were grown to OD600 of 2.7, then concentrated and rendered electrocompetent.

During cell growth, a second editing vector was prepared in the isothermal nucleic acid assembly module. The second editing vector comprised a kanamycin resistance gene, and the editing cassette comprised a galK Y145* edit. If successful, the galK Y145* edit confers on the cells the ability to uptake and metabolize galactose. The edit generated by the galK Y154* cassette introduces a stop codon at the 154th amino acid reside, changing the tyrosine amino acid to a stop codon. This edit makes the galK gene product non-functional and inhibits the cells from being able to metabolize galactose. Following assembly, the second editing vector product was de-salted in the isothermal nucleic acid assembly module using AMPure beads, washed with 80% ethanol, and eluted in buffer. The assembled second editing vector and the electrocompetent E. Coli cells (that were transformed with and selected for the first editing vector) were transferred into a transformation module for electroporation, using the same parameters as detailed above. Following electroporation, the cells were transferred to a recovery module (another growth module), allowed to recover in SOC medium containing carbenicillin. After recovery, the cells were held at 4° C. until retrieved, after which an aliquot of cells were plated on LB agar supplemented with chloramphenicol, and kanamycin. To quantify both lacZ and galK edits, replica patch plates were generated on two media types: 1) MacConkey agar base supplemented with lactose (as the sugar substrate), chloramphenicol, and kanamycin, and 2) MacConkey agar base supplemented with galactose (as the sugar substrate), chloramphenicol, and kanamycin. All liquid transfers were performed by the automated liquid handling device of the automated multi-module cell processing system.

In this recursive editing experiment, 41% of the colonies screened had both the lacZ and galK edits, the results of which were comparable to the double editing efficiencies obtained using a “benchtop” or manual approach.

### Example VIII: Enhanced Sensitivity and Tolerance to p-Coumaric Acid with Modified rpoA

To illustrate steps of a process for generating a cell population having a tolerance and/or a sensitivity to a compound p-coumaric acid was chosen to assay responses against the RNA alpha subunit encoded by the gene rpoA. A saturation mutagenesis library was generated for rpoA that covered each amino acid with all 19 other amino acids at each position, with a Onyx® unique barcode was used to identify the behavior of each design in the library. Using the results from the individual saturation mutagenesis library, a second and third library were generated which was a nearby-in-sequence combinatorial library that generated by the enrichment or depletion effect in the first selection experiment with the saturation mutagenesis library.

This library included 7,500 designs with combinatorial mutations of identified amino acid changes that gave the edited cell sensitivity or tolerance to p-coumaric acid. The combinatorial mutation library was then grown for 18 hours at 30 C in Loria broth (LB) supplemented with chloramphenicol (68 ug/L) and carbenicillin (1 mg/L) followed by growth in Minimal Media supplemented with glucose and antibiotics for 24 hours, and then split in triplicated and subjected to a titrated amount of p-coumaric acid (1 mg/L, 2 mg/L, 3 mg/L) along with an equal volume control triplicate of DMSO, each in minimal media with antibiotics. After 24 hours in selective conditions, the cells were harvested and selected for a first sub-population of cells having a tolerance to p-coumaric acid and a second sub-population of cells having a sensitivity to p-coumaric acid; thus generating the cell population having a tolerance to the first compound and the sensitivity to the second compound. In this particular examples, p-coumaric acid served as both the first compound (e.g., screened to provide tolerance) and the second compound (e.g., screened to provide sensitivity), but different compounds can be used in alternative embodiments. Subsequently, the Onyx® barcode diversity assay was executed to count the barcodes present in the population and identify which designs are enriching or depleting.

Enrichment was calculated by observing the log design representation of design in the selection condition over the design representation of the equivalent design in the control condition. The list was then sorted by most and least enriching and the associated edit was matched to identify engineered designs in rpoA which yield enhanced sensitivity or enhanced tolerance to p-coumaric acid.

Table 3 illustrates exemplary mutations observed on a test gene, rpoA, that led to increased tolerance to cell growth in the presence of coumaric acid.

TABLE 3

| | | Amino Acid |
|---|---|---|
| | | substitutions |
| | | WildType (amino |
| | Identified nucleotide | acids) |
| Gene | substitution edit sequence | Edited Sequence |
| rpoA | (181-225)_ATTGATGGTGTACTAC | YSTKE (SEQ ID |
| | ATGAGCGTATGAACAAAAAGGGCGTT | NO: 8)_(68-72) |
| | CAG (SEQ ID NO: 7) | RMNKK (SEQ ID |
| | | NO: 9) |
| rpoA | (682-726)_CTGGAAGCTTGGGTTG | FVDLRDVR (SEQ ID |
| | ACTTACGTTGGGTATATCAGCCTGAA | NO: 11) (231-238) |
| | GTG (SEQ ID NO: 10) | WVDLRWVY (SEQ |
| | | ID NO: 12) |
| rpoA | (685-729)_GAAGCTTGGGTTAAGT | FVDLRDVR (SEQ ID |
| | TACGTGATGTATATCAGCCTGAAGTG | NO: 11) (231-238) |
| | AAA (SEQ ID NO: 13) | WVKLRDVY (SEQ |
| | | ID NO: 14) |
| rpoA | (640-684)_GAAGAGGCGATTCGTA | RAATILAEQ (SEQ |
| | CCGCGATCCCGAGTCTGGCTCGTTGT | ID NO: 16) (219-227) |
| | CTG (SEQ ID NO: 15) | TAIPSLARC (SEQ ID |
| | | NO: 17) |
| rpoA | (181-225)_ATTGATGGTGTACTAC | YSTKE (SEQ ID |
| | ATGAGCGCAGCAACAAACGCGGCGTT | NO: 8) (68-72) |
| | CAG (SEQ ID NO: 18) | RSNKR (SEQ ID |
| | | NO: 19) |
| rpoA | (568-612)_TATAGCCCGCTGCAGG | ARVEQRT (SEQ ID |
| | GCATTGACCTGGACAAGCTGGTCATC | NO: 21) (190-196) |
| | GAA (SEQ ID NO: 20) | YSPLQGI (SEQ ID |
| | | NO: 22) |
| rpoA | (637-681)_CCTGAAGAGGCGATTC | RAATILAEQ (SEQ |
| | GTACCGCGTATACCGATCTGGCTCGT | ID NO: 16) (219-227) |
| | TGT (SEQ ID NO: 23) | TAYTDLARC (SEQ |
| | | ID NO: 24) |
| rpoA | (676-720)_TGGCAACTGGAAGCTT | FVDLRDVR (231- |
| | GGGTTAAATTACGTTATGTATATCAG | 238) |
| | CCT (SEQ ID NO: 25) | WVKLRYVY (SEQ |
| | | ID NO: 26) |
| rpoA | (640-684)_GAAGAGGCGATTCGTC | ATILAEQ (SEQ ID |
| | GTGCGATTCAGCGTCTGGCTCATAAA | NO: 28) (221-227) |
| | CTG (SEQ ID NO: 27) | IQRLAHK (SEQ ID |
| | | NO: 29) |
| rpoA | (691-735)_TGGGTTAAGTTACGTG | FVDLRDVR (SEQ ID |
| | ATGTATATCAGCCTGAAGTGAAAGAA | NO: 11) (231-238) |
| | GAG (SEQ ID NO: 30) | WVKLRDVY (SEQ |
| | | ID NO: 14) |
| rpoA | (640-684)_GAAGAGGCGATTCGTA | RAATILAE (SEQ ID |
| | CGGCGTTTCAGAAACTGGCTCGCCAA | NO: 32) (220-226) |
| | CTG (SEQ ID NO: 31) | TAFQKLAR (SEQ ID |
| | | NO: 33) |
| rpoA | (676-720)_GAAGAACTGGAAGCTT | QLEAFVDLRDVR |
| | GGCCGAAATTACGTTACGTATATCAG | (SEQ ID NO: 35) |
| | CCT (SEQ ID NO: 34) | (227-238) |
| | | ELEAWPKLRYVY |
| | | (SEQ ID NO: 36) |

Table 4 illustrates exemplary mutations observed on a test gene, rpoA, that led to increased sensitivity to cell growth in the presence of coumaric acid.

TABLE 4

| | Identified | Amino Acid substitutions |
|---|---|---|
| | nucleotide | WildType Sequence |
| | substitution | (amino acids) |
| Gene | edit sequence | Edited Sequence |
| rpoA | (814-858) | AEAIHYIGDLVQRTEVELLKTPN |
| | ACGGAATATATCCAC | (SEQ ID NO: 38)(272-294) |
| | TATATCGCAGATCTG | TEYIHYIADLVQREEVELLKTPN |
| | GTACAGCGTGAAGAG | (SEQ ID NO: 39) |
| | (SEQ ID NO: 37) | |
| rpoA | (907-951) | TEIKDVLASRGLSLGMRLENWPPA |
| | ATTAAAGACAAATGC | SIADE (SEQ ID NO: 41) |
| | GCTTCCCGTGGACGT | (301-328)_TEIKDKCASRGRSA |
| | TCTGCGACGATGCGC | TMRLENWPPASIADE |
| | (SEQ ID NO: 40) | (SEQ ID NO: 42) |
| rpoA | (913-957)_ | TEIKDVLASRGLSLGMRLENWPPA |
| | GACAAAACGGCTTCC | SIADE (SEQ ID NO: 41) |
| | CGTGGACGCTCTCTG | (301-328)_TEIKDKTASRGRSL |
| | GGCCCGCGCCTGGAA | GPRLENWPPASIADE |
| | (SEQ ID NO: 43) | (SEQ ID NO: 44) |
| rpoA | (904-948)_ | TEIKDVLASRGLSLGMRLENWPPA |
| | GATATTAAAGACAAA | SIADE (SEQ ID NO: 41) |
| | ACCGCTTCCCGTGGA | (301-328)_TDIKDKTASRGLSL |
| | CTGTCTCTGATTCCA | IPRLENWPPASIADE |
| | (SEQ ID NO: 45) | (SEQ ID NO: 46) |
| rpoA | (838-882)_ | AEAIHYIGDLVQRTEVELLKTPN_ |
| | GATCTGGTACAGAAA | (SEQ ID NO: 38) |
| | GAGGAGACCGAGCTC | (272-294)_AEAIHYIGDLVQKE |
| | CTTAAAGTTCCTCAG | ETELLKVPQL |
| | (SEQ ID NO: 47) | (SEQ ID NO: 48) |

The selection of a sub-population of cells (e.g., tolerant) can be performed in parallel (OR) with the selection of the second sub-population of cells. The selection of a sub-population of cells can also be performed in series (AND) with the selection of the second sub-population of cells. Example VI describes methodologies for recursive editing, where AND and OR populations for various compounds can be identified in a recursive manner.

### Example IX: Identification of Edited Transcription Factor Genes that Enable Boolean Sorts from a Population

To identify edits that can be utilized as Logic Gate Selection Markers, a library was generated to perturb transcription response by introducing edits to various transcription factors. The library was generated such that individual edits were introduced into individual cells for upregulating and inducing constitutive expression of the transcription factor by two medium-strength promoters known in the art (TTTACGGCTAGCTCAGTCCTAGGTATAGTGCTAGCTATGTTGGTA (SEQ ID NO: 49), TTGACGGCTAGCTCAGTCCTAGGTACAGTGCTAGCCATTCAGTGA (SEQ ID NO:50)) at position 16 nucleotides from the 5′ position of the coding sequence (CDS) start position, thus leaving the native ribosome binding site and other promoters intact; and separating two knock-out edits by introducing three stop codons in tandem in the 10th amino acid position of the CDS. Finally, a fifth edit was made to each transcription factor by truncating the transcription factor 50% of the way into the gene by nucleotide count, with the stop codon being introduced into each reading frame (TAATTAATTAA (SEQ ID NO: 51)).

The library was generated using the Onyx® platform technology to introduce the edits. This library was then grown in Luria broth for 18 hours in the presence of 25 ug/L chloramphenicol and 1 mg/L carbenicillin in a baffled shake flask. The library was then equilibrated in minimal media supplemented with glucose for a further 24 hours. The library was then split into triplicate conditions that included titrating amounts of resveratrol or p-coumaric acid, brought up in neat DMSO, and a control triplicate was additionally grown in DMSO supplementation alone.

The titrated amounts of resveratrol were 0.114 mg/L and 0.228 mg/L. The titrated amounts of p-coumaric acid were 1 mg/L, 2 mg/L, and 3 mg/L. After 24 hours of growth, the cells were harvested and the Onyx Barcode Diversity Assay was used to quantify the number of counts that a specific barcode associated with each design was represented in the selected and control populations. The Barcode Design Representation was then calculated for each and enrichers or depletors in the population were identified by comparison of the log of the representation of a design in the selected population over the same design in the control population. Triplicate measures gave insight into the reliability of the measurement. The enrichers and depletors were sorted in each experiment to obtain the highest enrichers and the highest depletors under each selective condition. Transcription factors in the table indicate those which were enriched or depleted most significantly in one compound, the other, or both.

The following candidates were identified when selecting for candidates that enriched and depleted in the exemplary compounds of resveratrol and coumaric acid.

TABLE 5 illustrates transcription factors edit enriching in coumaric acid and enriching in resveratrol (AND strains).

TABLE 5
Transcription factors edit enriching in
coumaric acid and enriching in resveratrol
(AND strains)

| Gene | Edit | Consequence |
|---|---|---|
| rob | TTGACGGCTAGCTCAGTCCTAGGTATTGTGC | Constitutive |
| | TAGCTATGTTGGTA (SEQ ID NO: 52) | Expression |
| | (constitutive promoter) inserted | |
| | 16 nucleotides 5′ of the CDS | |
| lrhA | TTTACGGCTAGCTCAGTCCTAGGTATAGTGC | Constitutive |
| | TAGCTATGTTGGTA (constitutive | Expression |
| | promoter) inserted 16 | |
| | nucleotides 5′ of the CDS | |

TABLE 6 illustrates transcription factors edit enriching in coumaric acid and depleting in resveratrol (OR strains).

TABLE 6
Transcription factors edit enriching in
coumaric acid and depleting in resveratrol
(OR strains)

| Gene | Edit | Consequence |
|---|---|---|
| hipB | TAATAATAA (stop codons; knock | Knockout |
| | out) substituted for nucleotides | |
| | 31-39 from the 5′ position | |
| lsrR | TAATAATAA (stop codons; knock | Knockout |
| | out) substituted for nucleotides | |
| | 31-39 from the 5′ position | |

TABLE 7 illustrates transcription factors edit depleting in coumaric acid and enriching in resveratrol (OR strains)

TABLE 7
Transcription factors edit depleting in
coumaric acid and enriching in resveratrol
(OR strains)

| Gene | Edit | Consequence |
|---|---|---|
| melR | TTGACGGCTAGCTCAGTCCTAGG | Constitutive |
| | TATTGTGCTAGCTATGTTGGTA | Expression |
| | (constituive promoter) | |
| | inserted 16 nucleotides 5′ | |
| | of the CDS | |
| dicA | TTGACGGCTAGCTCAGTCCTAGG | Constitutive |
| | TATTGTGCTAGCTATGTTGGTA | Expression |
| | (constituive promoter) | |
| | inserted 16 nucleotides 5′ | |
| | of the CDS | |

TABLE 8 illustrates transcription factors edit depleting in coumaric acid and depleting in resveratrol (AND strains)

TABLE 8
Transcription factors edit depleting in
coumaric acid and depleting in resveratrol
(AND strains)

| Gene | Edit | Consequence |
|---|---|---|
| gcvA | TTGACGGCTAGCTCAGTCCTAGG | Constitutive |
| | TATTGTGCTAGCTATGTTGGTA | Expression |
| | (constituive promoter) | |
| | inserted 16 nucleotides 5′ | |
| | of the CDS | |
| rpoS | TAATAATAA (stop codons; | Knockout |
| | knock out) substituted | |
| | for nucleotides 31-39 | |
| | from the 5′ position | |

Thus, a transcription factor such as rob or 1rhA when placed under constitutive expression were found to enrich over the control in both coumaric acid and resveratrol selection, while gcvA and rpoS were found to deplete in both coumaric acid and resveratrol selections compared to control conditions. Separately, dicA under constitutive expression, for example, was found to enrich significantly in resveratrol but deplete significantly in coumaric acid.

Thus, the edits in these sets of genes gives a knob to tune that allows chemical selective responses that can be identified by the assay described to allow for the generation of an organism that can have a response to one or more than one compounds. Further, the reproducibility of the transcription factor library response in the presence of the chemical gives a key signature of that chemical that is distinct or unique to the compound.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

We claim:

1. A method for Boolean storage of information in a cell population comprising:
transforming a cell population genetically engineered to have a tolerance to a first compound and a sensitivity to a second compound with a plurality of nucleic acids encoding at least two digital information files, thereby storing the at least two digital information files in a transformed cell population having Boolean logic for information retrieval.

2. The method of claim 1, further comprising growing the cell population having at least two digital information files in the first compound and in the second compound in parallel (OR) for selecting cell populations having Boolean selections of one or more cells encoding at least two digital information files.

3. The method of claim 1, further comprising growing the cell population having at least two digital information files in the first compound and in the second compound in series (AND) for selecting cell populations having Boolean selections of one or more cells encoding the at least two digital information files.

4. The method of claim 1, wherein the cell population is engineered to have a tolerance to at least 2 or more compounds.

5. The method of claim 1, wherein the cell population has a tolerance to a compound selected from the group consisting of coumaric acid, isobutryic acid, furfural, vanillin, 5-hydroxymethyl-furfural, NaCl, resveratrol, or syringic acid.

6. The method of claim 1, wherein the cell population is engineered to have a sensitivity to at least 2 or more compounds.

7. The method of claim 1, wherein the cell population has a sensitivity to a compound selected from the group consisting of coumaric acid, isobutryic acid, furfural, vanillin, 5-hydroxymethyl-furfural, NaCl, resveratrol, or syringic acid.

8. The method of claim 1, wherein the first compound is not an antibiotic.

9. The method of claim 1, wherein the second compound is not an antibiotic.

10. The method of claim 1, wherein at least two digital information files do not encode a component of a biological pathway.

11. The method of claim 1, further comprising retrieving the at least two digital information files from the cell populations having Boolean logic for information retrieval by sequencing.

12. The method of claim 11, wherein the sequencing is whole genome sequencing.

13. The method of claim 11, wherein the sequencing is a sequencing of a plasmid.

14. The method of claim 11, wherein each cell in the cell population comprises a barcode for sequencing at least one digital information file from the at least two digital information files.

15. The method of claim 1, wherein the plurality of nucleic acids encoding the exogenous digital information file can encode up to 50 million gigabytes, up to 100 million gigabytes, up to 150 million gigabytes, or up to 200 million gigabytes of the digital information.

16. The method of claim 15, wherein the plurality of nucleic acids encode a plurality of exogenous digital information files.

17. The method of claim 16, wherein the plurality of nucleic acids encode a plurality of exogenous digital information files encoding redundant information.

18. The method of claim 16, wherein the plurality of nucleic acids encode a plurality of exogenous digital information files encoding distinct information.

19. The method of claim 1, wherein the exogenous digital information file is first written as a sequence of 0s and 1s, and these 0s and 1s are written in the plurality of nucleic acids with the nucleotide bases A and C encoding for 0 and the nucleotide bases G and T encoding for 1.

20. The method of claim 1, wherein the exogenous digital information file is first written as a sequence of 0s and 1s, and these 0s and 1s are written in the plurality of nucleic acids with the nucleotide bases A and C encoding for 1 and the nucleotide bases G and T encoding for 0.

21. The method of claim 1, wherein the exogenous digital information file is first written as a sequence of 0s and 1s, and these 0s and 1s are written in the plurality of nucleic acids with the nucleotide bases A and G encoding for 0 and the nucleotide bases C and T encoding for 1.

22. The method of claim 1, wherein the exogenous digital information file is first written as a sequence of 0s and 1s, and these 0s and 1s are written in the plurality of nucleic acids with the nucleotide bases G and C encoding for 1 and the nucleotide bases A and T encoding for 1.

23. The method of claim 1, wherein the cell population is a population of bacterial cells.

24. The method of claim 1, wherein the cell population is genetically engineered to have the tolerance to the first compound and the sensitivity to the second compound by genome wide editing with a library of CRISPR editing cassettes.

25. The method of claim 24, wherein the library of gene editing cassettes is a library of randomly designed CRISPR editing cassettes.

26. The method of claim 24, wherein the library of gene editing cassettes is a library of rationally designed CRISPR editing cassettes.

27. The method of claim 24, wherein the CRISPR editing cassettes are CREATE gene editing cassettes comprising:
(a) a nucleic acid encoding a guide RNA (gRNA) comprising a region complementary to a target region of a nucleic acid in one or more cells of the cell population;
(b) a region homologous to the target region in the one or more cells of the cell population having a change in sequence of at least one nucleotide relative to a wild type reference genome of the cell; and
(c) a region comprising a site conferring immunity to nuclease-mediated editing.

28. A population of cells having Boolean logic for information retrieval as described in claim 1.

29. A method of environmental biosensing comprising:
contacting a sample from an environment to a cell population genetically engineered to have a tolerance to a plurality of compounds, wherein one or more cells in the cell population are associated with at least one barcode, whereby each barcode is a unique identifier for a compound in the plurality of compounds;
enriching for one or more cells in the cell population by growing the one or more cells in the presence of the sample from the environment, thereby generating a sample tolerant population; and
sequencing the at least one barcode from one or more cells in the sample tolerant population thereby generating a sequencing file that identifies a cell having a tolerance to the sample, whereby the identification of the tolerant cell provides for environmental biosensing.

30. The method of claim 29, wherein the cell population is genetically engineered to have a tolerance to at least 5 compounds, at least 100 compounds, or at least 500 compounds.

31. The method of claim 29, wherein the cell population is genetically engineered to have a tolerance to a first pre-determined compound.

32. The method of claim 31, wherein the first pre-determined compound is selected from the group consisting of coumaric acid, isobutryic acid, furfural, vanillin, 5-hydroxymethyl-furfural, NaCl, resveratrol, or syringic acid.

33. The method of claim 31, wherein the first pre-determined compound is an environmental pollutant.

34. The method of claim 31, wherein the first pre-determined compound is not an antibiotic.

35. The method of claim 29, further wherein the cell population is genetically engineered to have a tolerance to a plurality of compounds have a sensitivity to a plurality of compounds.

36. The method of claim 35, wherein the cell population is genetically engineered to have a sensitivity to at least 5 compounds, at least 100 compounds, or at least 500 compounds.

37. The method of claim 35, wherein the cell population is genetically engineered to have a sensitivity to a second pre-determined compound.

38. The method of claim 37, the second pre-determined compound is selected from the group consisting of coumaric acid, isobutryic acid, furfural, vanillin, 5-hydroxymethyl-furfural, NaCl, resveratrol, or syringic acid.

39. The method of claim 37, wherein the second pre-determined compound is an environmental pollutant.

40. The method of claim 37, wherein the second pre-determined compound is not an antibiotic.

41. The method of claim 29, further comprising comparing the growth of the sample tolerant population against a population fingerprint, wherein the population fingerprint is a reference population growth behavior in the presence of a known compound.

42. The method of claim 29, wherein the cell population is a population of bacterial cells.

43. The method of claim 29, wherein the cell population is genetically engineered to have the tolerance to the plurality of compounds by genome wide editing with a library of CRISPR editing cassettes.

44. The method of claim 43, wherein the library of gene editing cassettes is a library of randomly designed CRISPR editing cassettes.

45. The method of claim 43, wherein the library of gene editing cassettes is a library of rationally designed CRISPR editing cassettes.

46. The method of claim 43, wherein the CRISPR editing cassettes are CREATE gene editing cassettes comprising:
(d) a nucleic acid encoding a guide RNA (gRNA) comprising a region complementary to a target region of a nucleic acid in one or more cells of the cell population;
(e) a region homologous to the target region in the one or more cells of the cell population having a change in sequence of at least one nucleotide relative to a wild type reference genome of the cell; and
a region comprising a site conferring immunity to nuclease-mediated editing.

47. A population of cells for environmental biosensing as described in claim 29.

48. A process for generating a cell population having a tolerance to a first compound and a sensitivity to a second compound using a CRISPR system, the process comprising:
editing the cell population with a library of CRISPR editing cassettes, wherein the CRISPR editing cassettes comprise:
(f) a nucleic acid encoding a guide RNA (gRNA) comprising a region complementary to a target region of a nucleic acid in one or more cells of the cell population;
(g) a region homologous to the target region in the one or more cells of the cell population having a change in sequence of at least one nucleotide relative to a wild type reference genome of the cell;
(h) optionally a region comprising a site conferring immunity to nuclease-mediated editing; and
selecting for:
a first sub-population of cells having a tolerance to the first compound by growing the cells in the presence of the first compound and isolating a tolerant first population; and
a second sub-population of cells having a sensitivity to the second compound by growing the cells in the presence of the second compound and isolating cells from a sensitive second population;
thus generating the cell population having a tolerance to the first compound and the sensitivity to the second compound.

49. The process of claim 48, wherein the selection of the first sub-population of cells is performed in parallel (OR) with the selection of the second sub-population of cells.

50. The process of claim 48, wherein the selection of the first sub-population of cells is performed in series (AND) with the selection of the second sub-population of cells.

51. The process of claim 48, wherein the library of gene editing cassettes is a library of CRISPR editing cassettes.

52. The process of claim 48, wherein the library of gene editing cassettes is a library of random CRISPR editing cassettes.

53. The process of claim 48, wherein the library of gene editing cassettes is a library of rationally designed CRISPR editing cassettes.


SEQUENCE LISTING (52 sequences)

SEQ ID NO: 1 - DNA, 61 bp, Artificial Sequence
  ON127SPG1
ttgacggctagctcagtcctaggtacagtgctagctaagtaggtacgtaaggaggtgataa

SEQ ID NO: 2 - DNA, 61 bp, Artificial Sequence
  ON127SPG2
ctgatagctagctcagtcctagggattatgctagctaagtaggtacgtaaggaggtgataa

SEQ ID NO: 3 - DNA, 61 bp, Artificial Sequence
  ON127SPG3
ttgacagctagctcagtcctagggactatgctagctaagtaggtacgtaaggaggtgataa

SEQ ID NO: 4 - DNA, 61 bp, Artificial Sequence
  ON127SPG4
tttacggctagctcagtcctaggtatagtgctagctaagtaggtacgtaaggaggtgataa

SEQ ID NO: 5 - DNA, 61 bp, Artificial Sequence
  ON127SPG5
tttacagctagctcagtcctaggtattatgctagctaagtaggtacgtaaggaggtgataa

SEQ ID NO: 6 - DNA, 61 bp, Artificial Sequence
  ON127SPG13
ttgacagctagctcagtcctaggtataatgctagctaagtaggtacgtaaggaggtgataa

SEQ ID NO: 7 - DNA, 45 bp, Artificial Sequence
  rpoA
attgatggtgtactacatgagcgtatgaacaaaaagggcgttcag

SEQ ID NO: 8 - protein, 5 aa, Artificial Sequence
  rpoA
YSTKE

SEQ ID NO: 9 - protein, 5 aa, Artificial Sequence
  rpoA
RMNKK

SEQ ID NO: 10 - DNA, 45 bp, Artificial Sequence
  rpoA
ctggaagcttgggttgacttacgttgggtatatcagcctgaagtg

SEQ ID NO: 11 - protein, 8 aa, Artificial Sequence
  rpoA
FVDLRDVR

SEQ ID NO: 12 - protein, 8 aa, Artificial Sequence
  rpoA
WVDLRWVY

SEQ ID NO: 13 - DNA, 45 bp, Artificial Sequence
  rpoA
gaagcttgggttaagttacgtgatgtatatcagcctgaagtgaaa

SEQ ID NO: 14 - protein, 8 aa, Artificial Sequence
  rpoA
WVKLRDVY

SEQ ID NO: 15 - DNA, 45 bp, Artificial Sequence
  rpoA
gaagaggcgattcgtaccgcgatcccgagtctggctcgttgtctg

SEQ ID NO: 16 - protein, 9 aa, Artificial Sequence
  rpoA
RAATILAEQ

SEQ ID NO: 17 - protein, 9 aa, Artificial Sequence
  rpoA
TAIPSLARC

SEQ ID NO: 18 - DNA, 45 bp, Artificial Sequence
  rpoA
attgatggtgtactacatgagcgcagcaacaaacgcggcgttcag

SEQ ID NO: 19 - protein, 5 aa, Artificial Sequence
  rpoA
RSNKR

SEQ ID NO: 20 - DNA, 45 bp, Artificial Sequence
  rpoA
tatagcccgctgcagggcattgacctggacaagctggtcatcgaa

SEQ ID NO: 21 - protein, 7 aa, Artificial Sequence
  rpoA
ARVEQRT

SEQ ID NO: 22 - protein, 7 aa, Artificial Sequence
  rpoA
YSPLQGI

SEQ ID NO: 23 - DNA, 45 bp, Artificial Sequence
  rpoA
cctgaagaggcgattcgtaccgcgtataccgatctggctcgttgt

SEQ ID NO: 24 - protein, 9 aa, Artificial Sequence
  rpoA
TAYTDLARC

SEQ ID NO: 25 - DNA, 45 bp, Artificial Sequence
  rpoA
tggcaactggaagcttgggttaaattacgttatgtatatcagcct

SEQ ID NO: 26 - protein, 8 aa, Artificial Sequence
  rpoA
WVKLRYVY

SEQ ID NO: 27 - DNA, 45 bp, Artificial Sequence
  rpoA
gaagaggcgattcgtcgtgcgattcagcgtctggctcataaactg

SEQ ID NO: 28 - protein, 7 aa, Artificial Sequence
  rpoA
ATILAEQ

SEQ ID NO: 29 - protein, 7 aa, Artificial Sequence
  rpoA
IQRLAHK

SEQ ID NO: 30 - DNA, 45 bp, Artificial Sequence
  rpoA
tgggttaagttacgtgatgtatatcagcctgaagtgaaagaagag

SEQ ID NO: 31 - DNA, 45 bp, Artificial Sequence
  rpoA
gaagaggcgattcgtacggcgtttcagaaactggctcgccaactg

SEQ ID NO: 32 - protein, 8 aa, Artificial Sequence
  rpoA
RAATILAE

SEQ ID NO: 33 - protein, 8 aa, Artificial Sequence
  rpoA
TAFQKLAR

SEQ ID NO: 34 - DNA, 45 bp, Artificial Sequence
  rpoA
gaagaactggaagcttggccgaaattacgttacgtatatcagcct

SEQ ID NO: 35 - protein, 12 aa, Artificial Sequence
  rpoA
QLEAFVDLRDVR

SEQ ID NO: 36 - protein, 12 aa, Artificial Sequence
  rpoA
ELEAWPKLRYVY

SEQ ID NO: 37 - DNA, 45 bp, Artificial Sequence
  rpoA
acggaatatatccactatatcgcagatctggtacagcgtgaagag

SEQ ID NO: 38 - protein, 23 aa, Artificial Sequence
  rpoA
AEAIHYIGDLVQRTEVELLKTPN

SEQ ID NO: 39 - protein, 23 aa, Artificial Sequence
  rpoA
TEYIHYIADLVQREEVELLKTPN

SEQ ID NO: 40 - DNA, 45 bp, Artificial Sequence
  rpoA
attaaagacaaatgcgcttcccgtggacgttctgcgacgatgcgc

SEQ ID NO: 41 - protein, 29 aa, Artificial Sequence
  rpoA
TEIKDVLASRGLSLGMRLENWPPASIADE

SEQ ID NO: 42 - protein, 29 aa, Artificial Sequence
  rpoA
TEIKDKCASRGRSATMRLENWPPASIADE

SEQ ID NO: 43 - DNA, 45 bp, Artificial Sequence
  rpoA
gacaaaacggcttcccgtggacgctctctgggcccgcgcctggaa

SEQ ID NO: 44 - protein, 29 aa, Artificial Sequence
  rpoA
TEIKDKTASRGRSLGPRLENWPPASIADE

SEQ ID NO: 45 - DNA, 45 bp, Artificial Sequence
  rpoA
gatattaaagacaaaaccgcttcccgtggactgtctctgattcca

SEQ ID NO: 46 - protein, 29 aa, Artificial Sequence
  rpoA
TDIKDKTASRGLSLIPRLENWPPASIADE

SEQ ID NO: 47 - DNA, 45 bp, Artificial Sequence
  rpoA
gatctggtacagaaagaggagaccgagctccttaaagttcctcag

SEQ ID NO: 48 - protein, 24 aa, Artificial Sequence
  rpoA-
AEAIHYIGDLVQKEETELLKVPQL

SEQ ID NO: 49 - DNA, 45 bp, Artificial Sequence
  Promoter
tttacggctagctcagtcctaggtatagtgctagctatgttggta

SEQ ID NO: 50 - DNA, 45 bp, Artificial Sequence
  Promoter
ttgacggctagctcagtcctaggtacagtgctagccattcagtga

SEQ ID NO: 51 - DNA, 11 bp, Artificial Sequence
  Gene
taattaattaa

SEQ ID NO: 52 - DNA, 45 bp, Artificial Sequence
  constitutive promoter
ttgacggctagctcagtcctaggtattgtgctagctatgttggta